(19) United States
(12) Patent Application Publication
(10) Pub. No.: US 2025/0366785 A1
(43) Pub. Date: Dec. 4, 2025

(54) METHODS FOR PROCESSING, ENRICHMENT, DELIVERY, FORMULATION, AND UPTAKE FOR SUPPLEMENTS AND PHARMACEUTICALS

(71) Applicant: Russell van de Casteele, Pampano Beach, FL (US)

(72) Inventor: Russell van de Casteele, Pampano Beach, FL (US)

(21) Appl. No.: 18/874,457

(22) PCT Filed: Jun. 13, 2023
(86) PCT No.: PCT/US2023/068341

§ 371 Date: Dec. 12, 2024

Related U.S. Application Data
(60) Provisional application No. 63/366,275, filed on Jun. 13, 2022.

(51) Int. Cl. A61B 5/00; A61B 5/024; A61K 9/00; A61K 9/107; A61K 31/122; A61K 31/355; A61K 31/593; A61K 31/714; B01F 23/411; B01F 31/80; B01F 31/81; B01F 33/301; B01F 35/71; B01F 101/22

(52) U.S. Cl. CPC A61B 5/4848; A61B 5/02405; A61K 9/006; A61K 9/1075; A61K 31/122; A61K 31/355; A61K 31/593; A61K 31/714; B01F 23/4111; B01F 31/81; B01F 31/89; B01F 33/301; B01F 35/712; B01F 35/7176; B01F 35/718051; B01F 2101/22; B01F 2215/0431; B01F 2215/0468; B01F 2215/0481

(57) ABSTRACT

A method for improving stability and/or absorption of one or more biologically active agents including preparing formulations wherein a biologically active agent is dispersed using a homogenizer and/or a nanofluidizer; and optionally: i) enriching the formulation; and/or ii) delivering the formulations to a subject whereby the biologically active agent is absorbed by said subject, and/or iii) testing the formulation to identify suitable dosing ranges using computational modeling of biomolecular pathways to determine at least one feature selected from absorption in a cell, saturation of a cell, and potential toxicity in a cell, and/or iv) testing the formulation by monitoring with a low impact, minimally intrusive heart rate variability monitoring to enable rapid determination of neurological and physiological effects of a dosage, establishing dosing levels of the biologically active agent, and/or v) defining the corresponding metabolic effects of the dosage of the biologically active agent on the subject.

### CROSS REFERENCE TO RELATED APPLICATIONS

This application claims the benefit of U.S. Provisional Application No. 63/366,275, filed on Jun. 13, 2022, the entire disclosure of which is hereby incorporated by reference as if set forth fully herein.

### FIELD OF THE INVENTION

The present invention relates to methods for the processing, enrichment, delivery, formulation, uptake and testing of biologically active agents useful as supplements and pharmaceuticals. These methods may include identifying suitable dosing ranges, providing enhanced formulation stability, improve delivery and effectiveness and/or promote uptake of the biologically active agents into the bloodstream and/or the cells as well as testing for these results. These results may be achieved via implementation of one or more of the elements of the methods described herein including, for example, nanofluidization techniques applied to the formulations and enhancements achieved by enrichment and other methods for delivery of the biologically active agents via transmucosal and/or transdermal pathways and application of test methods to confirm the results.

### BACKGROUND OF THE INVENTION

Biologically active agents such as nutritional supplements, hormones, and a variety of pharmaceutical preparations, which will generally be referred to as “biologically active agents' are typically provided in oral (liquids or solids) or injectable dosage formulations. However, there are many disadvantages associated with these types of administration and delivery.

A biologically active agent is a medicine, supplement or other substance which has a physiological effect when ingested or otherwise introduced into the body. Many biologically active agents may be degraded within the gastrointestinal (GI) tract and/or may undergo first-pass metabolism in the liver. In addition, there exists a large segment of the population that experience difficulty swallowing pills or are unable to tolerate ingestion of solids.

During the past three decades, however, formulations that control the rate and period of drug delivery (e.g., time-release medications) and target specific areas of the body for treatment have become increasingly common and complex. Some have provided additional options for administering certain types of biologically active agents but there are still a large number of supplements and medications that do not achieve maximum effect because they do not reach their intended targets either fast enough or in high enough concentrations, or, in some cases concentrations that are too high such that the effect may become toxic and cause side effects.

The potency and therapeutic effects of many biologically active agents that are orally administered are limited or reduced by the partial degradation that occurs before these biologically active agents reach their desired target in the body. Further, injectable medications may be less expensive and could be administered more easily if they were dosed by other routes such as absorption via the oral mucosa, the pulmonary mucosa, the vagina and the intestinal tract. In order to achieve this, it is necessary to develop formulations and methods suitable to safely administer biologically active agents through these specific areas of the body without toxicity. This can be complex since particular physiological environments (e.g. low pH in the stomach) can degrade a biologically active agent or may not be suitable for rapid and/or complete absorption. Also, in some cases biologically active agents cannot be administered via an area where healthy tissue could be adversely affected by the biologically active agent.

Transmucosal administration routes offer distinct advantages. Of the various routes, the mucosal linings of the nasal passages and the oral cavity are the most attractive due to their rapid and high levels of absorption. Although intranasal administration has been successful for several drugs, such as allergy medications, potentially serious side-effects, such as irritation and possible irreversible damage to the ciliary action of the nasal cavity from chronic application, have deterred health professionals from recommending long-term use of drugs via intranasal administration.

Within the oral cavity, there are three generally recognized routes of administration. Local delivery for applications involving treatment of a disorder within the oral cavity itself, such as a canker sore. Sublingual delivery via the mucosal membranes lining the floor of the mouth which provides rapid absorption and which is used for agents such as nitroglycerin, which is placed under the tongue for sublingual administration. The high permeability of the sublingual mucosa and the rich blood supply to the sublingual mucosa, transport via the sublingual route results in a rapid onset of action, providing a delivery route appropriate for highly permeable agents with short delivery time requirements and an infrequent dosing regimen. A drawback of sublingual delivery is that it produces a saliva wash (swallowing) and in the case of nitrolingual nitroglycerin spray, it has been found to cause headaches as a result of administering an excess of the drug needed to accomplish its' task.

The third generally recognized route of administration via the oral cavity is via the buccal mucosa. This area encompasses the mucosal membranes of the inner lining of the cheeks. This area also has a rich blood supply, is robust, and exhibits a short cellular recovery time following stress or damage. Although the buccal mucosa is less permeable than the sublingual mucosa, the expanse of smooth and relatively immobile buccal mucosa provides a highly desirable absorption pathway for sustained-release and controlled-release delivery of agents. As with other transmucosal routes of administration, two major advantages of this route are avoiding both hepatic first-pass metabolism and pre-systemic elimination within the GI tract.

One of the major disadvantages associated with delivery of agents via the buccal mucosa has been the relatively low passage of active agents across the mucosal epithelium, thereby resulting in low agent bioavailability, which translates into a substantial loss of the active agent present in the dosage form. Various permeation and absorption enhancers, such as POLYSORBATE-80™, sorbitol, and phosphatidylcholine have been explored to improve passage of drugs through the buccal mucosa. Studies have indicated that the superficial layers and protein domain of the epithelium may be responsible for maintaining the barrier function of the buccal mucosa (Gandhi and Robinson, Int. J. Pharm. (1992) 85, pp. 129-140).

It is known that use of a permeation enhancer can increase the passage of a biomolecule. Further, studies have suggested the feasibility of buccal delivery of even a rather high molecular weight pharmaceutical (Aungst and Rogers, Int. J. Pharm. (1989) 53, pp. 227-235).

Bioadhesive polymers have also been investigated for use in buccal delivery systems. Bioadhesive polymers have been developed to adhere to a biological substrate to maintain continuous contact of an agent with the site of delivery. This process has been termed “mucoadhesion” when the substrate is mucosal tissue (Ch'ng et al., J. Pharm. Sci. (1985) 74, 4, pp. 399-405).

A goal of delivery systems is to deploy intact agents to specifically targeted parts of the body through a medium that can control the administration by means of either a physiological or chemical trigger. To achieve this goal, a number of researchers have turned to advances in micro-and nanotechnology. One prominent area of endeavor is the production of so-called “nanoparticles” which act as chemical or physical “carriers” of biologically active agents.

During the past decade, novel polymeric microspheres, polymer micelles, and hydrogel materials have been shown to be effective in enhancing the specificity of drug targeting, lowering systemic drug toxicity, improving treatment absorption rates, and providing protection for pharmaceuticals against biochemical degradation. In addition, several other drug delivery systems show signs of promise, including those composed of biodegradable polymers, dendrimers, electroactive polymers, and modified C-60 fullerenes (also known as “buckyballs”).

Polymeric delivery systems are based on “carriers” which are composed of polymeric chemical compounds. These carriers are associated with agents to form complex, large molecules, which “carry” the agent across physiological barriers. Illustrative examples of these polymeric compounds include poly(ethylene-glycol)-poly(alpha, beta-aspartic acid), carboxylates, and heterobifunctional polyethylene glycol.

Carrier Proteins that transport solute molecules across lipid membrane bi-layers can act more efficiently when transporting molecular dispersions of the present invention. The transported solute is not covalently modified by the Carrier Protein but instead is delivered unchanged to the other side of the membrane. (Alberts B, Johnson A, Lewis J, et al. Molecular Biology of the Cell. 4th edition. New York: Garland Science; 2002.) The processed molecular dispersions of the present invention can more effectively be transported across the cell membrane by Carrier Proteins since less energy is required to move uniform molecular dispersions containing nanoparticles.

One type of nanotechnology involves the use of “hydrogels” as carriers of drugs. The principle behind this technology is to use a chemical compound which traps the active compound and then releases the active compound by swelling or expanding inside of specific tissues, thus allowing delivery of a high concentration of the active agent to the target while protecting degradable active agents during transit to the target. Hydrogels are specialized systems that are generally formulated to meet specific needs for the delivery of individual active agents.

During the past two decades, research into hydrogel delivery systems has focused primarily on systems containing polyacrylic acid (PAA) backbones. PAA hydrogels are known for their super-absorbency and ability to form extended polymer networks through hydrogen bonding. In addition, they are excellent bioadhesives, which means that they can adhere to mucosal linings within the body for extended periods, and they can be designed to release their encapsulated active agents slowly over time.

One example of the complexity of these systems is a glucose-sensitive hydrogel for delivery of insulin to diabetic patients using an internal pH trigger. This system features an insulin-containing “reservoir” formed by a poly[methacrylic acid-g-poly(ethylene glycol)]hydrogel membrane in which glucose oxidase has been immobilized. The membrane itself is housed between non-swelling, porous “molecular fences”.

Although these approaches are the focus of intense research, other processes are also under consideration, including aerosol inhalation devices, transdermal methodologies, forced pressure injectables, and biodegradable polymer networks designed specifically to transport gene therapies.

Another method to formulate active agents for delivery has been the use of nanosuspensions. Nanosuspensions provide the active agents in the form of very small nanoparticles and thus can be formulated into substantially uniform suspensions or dispersions which ensure delivery of the desired dosage. The use of commercial devices such as mill processors, microfluidizers and homogenizers has allowed the formulation of nanosuspensions of a variety active agents. Active agents provided in nanosuspensions can be wrapped in liposomes or made into micellar mixtures by mixing with appropriate chemical compounds.

A variety of avenues have been explored in an effort to produce viable, efficient means for drug delivery via the buccal mucosa. Such avenues include the use of liposomal carriers to enhance uptake or facilitate delivery; decreasing the particle size of carriers, and employing a physical matrix, such as a sponge, to retain an active agent in the buccal area.

What is lacking is a method for increasing the bioavailability of a biologically active agent, which may be administered via various routes, but particularly for administration via the buccal mucosa or mucosal membranes, which method also provides a stable product.

The advantages of nanosuspensions have so far not been exploited, since it is difficult achieve this particle size range. For example, this particle size range is only accessible to a limited extent using conventional grinding techniques (dry grinding in a ball mill, air jet grinding). Air jet milling provides powders with 100% of the particles smaller than approximately 25-50 μm in diameter, but these powders contain only a few percent of particles with diameters in the nanometer range. An example is the particle size distribution of the air-jet-ground drug RMKP 22 (4-[N-(2-hydroxy-2-methyl-propyl)-ethanolamine]-2,7-bis(cis-2,6-) measured with a laser diffractometer (LD). Although 100% of the particle diameters are smaller than 25 μm, only 8% of the particle diameters are in the range below 1000 nm, i.e. 92% of the particles have diameters greater than 1 μm. One could now assume that the nanometer fraction is separated off and the remaining particles are subjected to a new grinding process in order to obtain further nanoparticles. However, this is only possible to a limited extent because the progressive grinding process leads to increasingly perfect crystals with an increasing degree of comminution, which cannot be further comminuted afterwards by the maximum achievable grinding forces (P. List, Arzneimittelformlehre, Wissenschaftliche Verlagsgesellschaft Stuttgart, 1976). In summary, it can thus be stated that nanoparticles can be produced from drugs using conventional dry grinding technology and subsequent fractionation, but with one major disadvantage: loss of about 90% of the active ingredient.

Wet grinding was used as a further grinding technique (Sandell, E., floor plan of the pharmaceutical pharmacy, Govi-Verlag GmbH, Frankfurt am Main, 1962), for example using a Premier Mill (Sandell, op. Cit.) Or a ball or Perlmüble (Hagers Handbook of Pharmaceutical Practice, Springer-Verlag, Berlin, 1925). Use of the pearl mill results in a main population of particles with diameters in the nanometer range, however, there is still a significant proportion of particles with diameters above 1 μm. For the drug RMKP 22. RMKP 22 (Dispermat) without added surfactant and with the addition of 3% Tween 80™ ground in the bead mill, the diameter is already 50% of the surfactant-free sample at approx. 2 μm diameter, i.e. 50% of the particles are >2 μm in diameter.

Some of these micrometer sized particles can be attributed to agglomeration. As described in the literature (Sandell, op. Cit.; P. H. List, drug form theory, scientific publishing company mbH Stuttgart, 1976; Sucker, H, Speiser, P., Fuchs, P., Pharmaceutical Technology, George Thieme Verlag Stuttgart, 1978; Münzel, K., Büchi, J., Schultz, O.-E., Galenie internship, Scientific Publishing Company mbH Stuttgart, 1959) particles can aggregate in suspensions as a result of adding surfactants or general stabilizers (e.g. polyvinylpyrrolidone).

Another reduction in particle size in such mills is possible if the viscosity of the dispersion medium is increased, the speed must remain constant (W. Holley, dissertation, Friedrichs University of Karlsruhe, 1984; W. Holley, homogenizing with high pressure, low pressure, ultrasound and other techniques, Lecture 35th Annual Congress of the APV, Strasbourg, 1989). Usually this is also recommended by the mill manufacturers (e.g. Dyno-Mill, A. Bachoffen AG machine factory). Surfactant-stabilized Microparticles have also been patented (U.S. Pat. No. 5,246,707), which also contains iron particles within the microparticles.

U.S. Pat. No. 5,681,600 discloses a stable, liquid nutritional product and a method for its manufacture. Preparation of the product comprises forming a protein solution, a carbohydrate solution, and an oil blend to combine with an amount of a nutritional ingredient containing soy polysaccharide. Soy polysaccharide is essential as a stabilizer to maintain the components in solution, thereby avoiding the need for carrageenan, and avoiding the need to overfortify the amount of nutritional ingredient in the composition, owing to degradation over time. The combined solution is subjected to microfluidization as an alternative to homogenization.

U.S. Pat. No. 5,056,511 discloses a method for compressing, atomizing, and spraying liquid substances for inhalation purposes. The liquid substance is compressed under high pressure to reduce its volume. The released liquid is then atomized to cause the liquid substance to burst into particles in the size range of about 0.5 μm to about 10 μm, thereby forming a very fine cloud for direct inhalation by the end-user. This method is intended for immediate use and does not provide a product having long-term stability.

U.S. Pat. No. 4,946,870 discloses a film-forming delivery system, which requires at least one aminopolysaccharide, useful for delivery of pharmaceutical or therapeutic active agents to a desired topical or mucous membrane site. The active agent may be delivered by a gel, patch, sponge, or the like.

U.S. Pat. No. 5,891,465 discloses the delivery of a biologically active agent in a liposomal formulation for administration via the mouth. The phospholipid vesicles of the liposomal composition provide an increase in bioavailability of the biologically active agent in comparison to an oral dosage form. The liposomal composition, while reaching a submicron level for absorption into the bloodstream, nevertheless requires specific components to be provided within a narrow range of concentrations to enable the one or more bilayer forming lipids to achieve delivery through the mucosal lining.

U.S. Pat. No. 5,981,591 discloses a sprayable analgesic composition and method of use. The sprayable dosage includes one or more surfactants for facilitating absorption through the buccal mucosa of the mouth. The use of surfactants for increasing bioavailability is of limited value, since they are only effective for a small proportion of biologically active agents.

Drug preparations called nanosuspensions were produced by high-pressure homogenization, and are the subject of U.S. Pat. No. 5,858,410 to Muller.

Prior to the use of high-pressure homogenization, nanosuspensions were prepared by a pearl milling process, which was a longer process than pressure homogenization. This technology is the subject of U.S. Pat. No. 5,271,944 to Lee. A number of other methods have been used to prepare nanosuspensions with varying degrees of success including low energy agitators, turbine agitators, colloid mills, sonolators, orifices, media mills, rotor stator mixers and sonicators.

### SUMMARY OF THE INVENTION

The present invention is directed to a method for preparing formulations by utilizing one or more dispersion methods. These formulations are stable, uniform formulations containing submicron particles. The formulations can be in the form of emulsions, suspensions, dispersions and/or mixtures thereof. These stable formulations enable enhanced delivery of a biologically active agent into the bloodstream and/or cells.

For example, the formulations of the present invention may include:
a) emulsions—prepared by mixing immiscible liquids, and
b) dispersions including stable suspensions—prepared by mixing at least two c) nanosuspension components, the dispersed material and the dispersion medium. The dispersed material is preferably a solid phase of submicron particles, or a phase containing submicron particles. The dispersion medium may be, for example, a liquid in which the dispersed material is distributed.

In each of the above embodiments, the formulations include dispersed submicron particles that are not fully dissolved. Preferably, the dispersed material is in the form of a nanoparticle.

The formulations of the instant invention can be prepared using aqueous or organic solvents to form the dispersions, stable suspensions or emulsions. For preparation of the emulsions, known emulsifying agents can be employed.

The formulations of the instant invention can be delivered by way of a sprayer that sprays micro- and/or nano-droplet sprays, an aerosol, a tablet, a pill, a liquid, a suppository, a gel, or protein carrier. Delivery may be accomplished by parenteral, intrathecal, intravenous, transdermal, transmucosal, and any or all commonly recognized methods for supplement and drug delivery.

A nanofluidization technique may be employed for the production of formulations containing submicron molecular dispersions in aqueous, organic and/or oil-based mixtures for use as supplement and drug delivery systems. Nanofluidization can be defined as the application of extreme shear and impact forces for molecular dispersion in liquids without excess heat or the breaking of chemical bonds.

The instant process does not require encapsulation of the active agents in polymers or the use of hydrogels or other supporting or encapsulating substances. This process allows active agents to be sprayed as microdroplets. The particle sizes of the formulations are less than a micron. This has been verified using a Malvern Spraytec device that measures droplets from the fine mist sprayers ranging from 30 to 100 microns in size and thus can accommodate the molecular dispersions of the present formulations. Each micron sized spray droplet contains thousands of molecules (i.e. suspension or emulsion) for enhanced absorption.

The formulations of the instant invention may be effective in providing higher concentrations of an active agent in the bloodstream over a longer period of time as compared to other active agents administered in a similar manner, e.g. by a oral mucosal route, intestinal absorption, or the like. While not wishing to be bound to any particular theory of operation, it has been hypothesized that the formulations of the instant invention allow molecules to be delivered across tissue barriers at a faster and more consistent rate than, for example, comparable non-nanofluidized formulations.

In its broadest context, the method includes mixing together various aqueous and/or non-aqueous components, e.g. organic or inorganic components. Depending upon the solubility of the biologically active agent(s), a nanofluidizable mixture may be obtained by adding the active agent(s) to one or more of an aqueous media, an organic media, an oil-based media, or a crude emulsion which may contain a mixture of two or more of said media. The mixture may further contain various components such as flavorings, preservatives, surfactants, and permeation enhancers.

Nanofluidizing said mixture provides a means for the mixture to form a stable uniform emulsion or dispersion having submicron particles of the active agent dispersed therein. This nanofluidized formulation may provide for one or more improvements in the period of onset, bioavailability, absorptivity and controlled or extended-release capability of the product. Upon contact of the nanofluidized formulation with the body, e.g. with an area of the oral cavity including the mucosal membranes, the active agent is absorbed into the bloodstream in an amount sufficient to elicit a desired biological response.

Accordingly, it is one object of the instant invention to provide a biologically active agent as a stable, substantially uniform formulation. This is achieved by the use of a nanofluidization process. These formulations are effective for administration via various routes, and particularly via the oral mucosal membranes.

It is a further object of the instant invention to provide a formulation capable of providing a predetermined period of onset of the effect of the biologically active agent.

It is yet another object of the instant invention to provide stable formulations which comprise a dispersion, suspension, or emulsion of a biologically active agent.

It is a still further object of the invention to provide formulations that have enhanced bioavailability compared to other formulations when delivered via various routes of administration, particularly via the oral mucosal membranes.

It is a further object of the instant invention to provide formulations that are capable of sustained-release, extended release, or controlled-release.

The following sentences may describe certain aspects of the present invention
1. In a first aspect, the present invention relates to methods for improving stability and/or absorption of one or more biologically active agents comprising a step of:
preparing one or more formulations wherein the one or more biologically active agents is dispersed using one or more homogenizer(s) and/or one or more nanofluidizer(s); and optionally comprising one or more of the following steps:
i) enriching the one or more formulations; and/or
ii) delivering the one or more formulations by contacting the one or more formulations with a subject, whereby the one or more biologically active agents is absorbed by said subject, and/or
iii) testing the one or more formulations to identify suitable dosing ranges using computational modeling of biomolecular pathways to determine at least one feature selected from the group consisting of absorption of the one or more biologically active agents in a cell, saturation of the one or more biologically active agents in a cell, and potential toxicity of the one or more biologically active agents in a cell, as determined by using a computational systems biology platform, and/or
iv) testing the one or more formulations by monitoring the one or more formulations with a low impact, minimally intrusive Heart Rate Variability monitoring to enable rapid determination of neurological and physiological effects of an established or a proposed dosage levels of the one or more biologically active agents on the subject comprising applying adjusted levels of the biologically active agent to the subject and measuring heart rate variability,
establishing efficient and effective dosing levels of the one or more biologically active agents on the subject, and
defining the corresponding metabolic effects of the efficient and effective dosing levels of the one or more biologically active agents on the subject.
2. The method of sentence 1, wherein the method may include testing the one or more formulations to identify suitable dosing ranges carried out using computational modeling of biomolecular pathways to determine the absorption of the biologically active agents in a cell, or to determine the saturation of biologically active agents in a cell, or to determine the potential toxicity of biologically active agents in a cell, as determined by a computational system biology platform.
3. The method of any one of sentences 1-2, wherein the method may include testing the one or more formulations by monitoring effects of the formulation with a low impact, minimally intrusive Heart Rate Variability monitor and/or monitoring and/or measuring electric fields in the body to enable rapid determination of neurological and physiological effects of an established or a proposed dosage level of the one or more biologically active agents on the subject comprising applying adjusted levels of the biologically active agent to the subject and measuring heart rate variability and body electric fields,
establishing efficient and effective dosing levels of the one or more biologically active agents on the subject, and
defining the corresponding metabolic effects of the efficient and effective dosing levels of the one or more biologically active agents on the subject.
4. The method of any one of sentences 1-2, wherein the method comprises testing the one or more formulations remotely through real-time, non-contact vital sign visual optical monitoring to measure and track metabolic effects and/or absorption of the biologically active agents of enriched formulas in the subject.
5. The method of any one of sentences 1-4, wherein the homogenizer and/or nanofluidizer may provide shear processing, cavitation, and impact processing of the one or more formulations comprising the one or more biologically active agents, wherein the homogenizer and/or nanofluidizer provides a constant high pressure of from about 5,000 psi to 45,000 psi and each of the shear processing, cavitation, and impact processing of the one or more formulations is configured to be independently adjusted,
6. The method of sentence 5, wherein a shear processing time may be modified to be shorter or longer, cavitation may be adjusted by adjusting a nozzle size, and impact processing may be adjusted by initiating a reverse flow setup.
7. The method of any one of sentences 1-4, wherein the homogenizer may carry out sonochemical and sonomechanical processes on the mixture comprising the one or more biologically active agents by providing an active ultrasonic cavitation region comprising:
a first cylindrical section having a first diameter and a first length, and including an entrance surface having an entrance cross-sectional area;
a first transitional section acoustically coupled to the first cylindrical section having a first variable cross-section and a first transitional length;
a second cylindrical section acoustically coupled to the first transitional section and having a second diameter and a second length;
a second transitional section acoustically coupled to the second cylindrical section and having a second variable cross-section and a second transitional length; and
a third section acoustically coupled to the second transitional section and having a third length, and including an exit surface having an exit cross-sectional area; and
wherein a total length of the cavitation region is equal to a multiple of one-half of an acoustic wavelength in the mixture comprising the one or more biologically active agents accounting for phase velocity dispersion; and
the first transitional length of said first transitional section is less than a value of ln(N)/k where N is the ratio of the first and second diameters of the first and second cylindrical sections, respectively, and k is a wave number representing an angular frequency of ultrasonic vibrations divided by the speed of sound in the mixture comprising the one or more biologically active agents.
8. The method of any one of sentences 1-4, wherein the homogenizer and/or nanofluidizer may include:
a) a hollow member and a rotor that includes one or more blades positioned at least partially within the hollow member, wherein the rotor blades are rotationally driven and the hollow member is not rotationally driven in a first combining mode; and
b) a plurality of fins extending outwardly from the hollow member, wherein the hollow member is rotationally driven in a second combining mode.
9. The method of any one of sentences 1-4, wherein the homogenizer may include:
a) a hollow member and a rotor at least partially within the hollow member, wherein the rotor rotates in a first combining mode;
b) a plurality of fins extending outwardly from the hollow member, wherein the hollow member rotates in a second combining mode; and
c) an interlock assembly adapted to permit selectively operating the device in the first combining mode or the second combining mode, wherein the interlock assembly comprises a first one-way bearing mounted to the rotor and the hollow member, and the first one-way bearing is adapted to permit the rotor to rotate in a first direction independent of the hollow member in the first combining mode and to interlock the hollow member and the rotor so that the hollow member rotates with the rotor in a second opposite direction in the second combining mode.
10. The method of any one of sentences 1-4, wherein the homogenizer may include:
a) a hollow member and a rotor at least partially within the hollow member, wherein the rotor rotates in a first combining mode;
b) a plurality of fins extending outwardly from the hollow member, wherein the hollow member rotates in a second combining mode, and wherein the rotor has one or more blades and the hollow member has a plurality of windows defined therein in alignment with the rotor blades, and the windows are interposed between the fins.
11. The method of any one of sentences 1-4, wherein the homogenizer may include:
a) a hollow member and a rotor having one or more blades that rotate at least partially within the hollow member in a first combining mode to perform high-shear homogenization;
b) a plurality of fins extending outwardly from the hollow member, wherein the hollow member rotates in a second combining mode to perform low-shear mixing;
c) an actuator operably coupled to the rotor; and
d) an interlock assembly adapted to permit selectively operating the device in the first combining mode or the second combining mode, wherein the interlock assembly is adapted to permit the rotor to rotate independently of the hollow member, to restrict rotation of the hollow member in the first combining mode, and to interlock the hollow member and the rotor so that the hollow member rotates with the rotor in the second combining mode.
12. The method of any one of sentences 1-4, wherein the nanofluidizer may include a blend application configured to issue blend instructions based on a specified recipe; and
a nanofluidic mixer device, including:
   a nanofluidic mixer device housing with hinged articulated opening;
   a plurality of nanofluidic pumps disposed within the device housing;
   a plurality of nanofluidic valves disposed within the device housing;
   a nanofluidic dispenser at least partially extending through the device housing;
   a nanofluidic mixer chip disposed within the device housing and configured to receive and meter nanofluidic amounts of each of at least a first fluid, a second fluid, and an at least one third fluid, each fluid having a viscosity different from a viscosity of each of the other fluids;
   a mix controller disposed within the device housing and configured to electronically communicate with the blend application and receive blend application blend instructions therefrom; and
   a plurality of fluid pathways defined therein and contained within the device housing, the fluid pathways including a first fluid pathway providing fluid communication from a first fluid canister containing the first fluid to the nanofluidic mixer chip, a second fluid pathway providing fluid communication from a second fluid canister containing the second fluid to the nanofluidic mixer chip, a third fluid pathway providing fluid communication from a third fluid canister containing the at least one third fluid to the nanofluidic mixer chip, and a fourth fluid path way providing fluid communication from the nanofluidic mixer chip to the nanofluidic dispenser, the nanofluidic dispenser being configured to receive metered nanofluidic amounts of each of the first fluid, the second fluid, and at least one third fluid from the nanofluidic mixer chip for dispensing;
the mix controller being configured to: (i) communicate with the blend application and receive blend instructions, and (ii) based on the blend instructions received from the blend application:
(a) control each of the plurality of nanofluidic pumps and each of the plurality of nanofluidic valves and thereby control a system pressure within the nanofluidic mixer device, such that:
   (1) the first fluid is delivered to the panofluidic mixer chip,
   (2) the second fluid is delivered to the nanofluidic mixer chip,
   (3) the at least one third fluid is delivered to the nanofluidic mixer chip, and
   (4) each of the first fluid, second fluid, and the at least one third fluid are metered at nanofluidic amounts according to the recipe to provide a nanofluidic mixture of each of the first fluid, the second fluid, and the at least one third fluid; and
(b) control each of the plurality of nanofluidic pumps and each of the plurality of nanofluidic valves such that nanofluidic mixture is dispensed from the nanofluidic dispenser.
13. The method of any one of sentences 1-4, wherein the nanofluidizer may include:
   at least one nanofluidic pump;
   at least one nanofluidic valve;
   a nanofluidic dispenser;
   a nanofluidic mixer chip configured to receive and mix a nanofluidic amount of a first fluid with a nanofluidic amount of at least one second fluid to form a nanofluidic mixture, the first fluid having a viscosity different from a viscosity of the at least one second fluid;
   a plurality of fluid pathways defined therein, including a first fluid pathway providing fluid communication from a reservoir containing the first fluid to the nanofluidic mixer chip, a second fluid pathway providing fluid communication from a reservoir containing the at least one second fluid to the nanofluidic mixer chip, and a third fluid pathway providing fluid communication from the nanofluidic mixer chip and a nanofluidic dispenser, the nanofluidic dispenser being configured to receive the nanofluidic mixture from the nanofluidic mixer chip and dispense the nanofluidic mixture from the nanofluidic dispenser; and
   a mix controller configured to control each of the at least one nanofluidic pump and the at least one nanofluidic valve, such that:
      (1) the nanofluidic amount of the first fluid is delivered to the nanofluidic mixer chip,
      (2) the nanofluidic amount of the at least one second fluid is delivered to the nanofluidic mixer chip,
      (3) the nanofluidic mixer chip mixes the first fluid and the at least one second fluid to form the nanofluidic mixture, and
      (4) the nanofluidic mixture is dispensed from the nanofluidic dispenser.
14. The method of any one of sentences 1-4, wherein the nanofluidizer may include:
a housing;
a computer processor;
a plurality of liquid reservoirs disposed within the housing;
at least one nanofluidic mixer chip disposed within the housing and in fluid communication with the plurality of liquid reservoirs, the nanofluidic mixer chip configured to receive and convey fluids from the plurality of liquid reservoirs and mix the received fluids, the received fluids having different viscosities;
   one or more valves in communication with the computer processor and configured to dispense liquids from at least two of the plurality of liquid reservoirs into the nanofluidic mixer chip based on one or more signals from the computer processor; and
a dispenser in fluid communication with the at least one nanofluidic mixer chip and configured to dispense the mixed fluids.
15. The method of any one of sentences 1-4, wherein the nanofluidizer may include:
a software application configured to issue instructions based on a specified recipe; and
a nanofluidic device, including:
a nanofluidic device housing with hinged articulated opening;
a nanofluidic pump disposed within the device housing;
a nanofluidic valve disposed within the device housing;
a nanofluidic dispenser at least partially extending through the device housing;
a nanofluidic chip disposed within the device housing and configured to receive and meter a nanofluidic amount of a fluid;
a controller disposed within the device housing and configured to electronically communicate with the software application and receive instructions therefrom; and
a first fluid pathway defined therein and contained within the device housing, the first fluid pathway providing fluid communication from a fluid canister containing the fluid to the nanofluidic chip, and a second fluid pathway providing fluid communication from the nanofluidic chip to the nanofluidic dispenser, the nanofluidic dispenser being configured to receive metered nanofluidic amounts of the fluid from the nanofluidic chip for dispensing;
the controller being configured to: (i) communicate with the software application and receive instructions, and (ii) based on instructions received from the software application.
(a) control the nanofluidie pump and the nanofluidic valve to control a system pressure within the nanofluidic device, such that: (1) the fluid is delivered to the nanofluidic chip, (2) the fluid is metered at a nanofluidic amount according to the recipe to provide a nanofluidic volume of the fluid; and
(b) control each of the nanofluidic pump and the nanofluidic valve such that the nanofluidic volume of the fluid is dispensed from the nanofluidic dispenser.
16. The method of any one of sentences 1-4, wherein the nanofluidizer may include:
   a first nanofluidie channel;
   a second nanofluidic channel extending along the first nanofluidic channel; and
   a first array of islands separating the first nanofluidic channel from the second nanofluidic channel, wherein a boundary of the first nanofluidic channel is defined by a first undulating outer wall, each island is separated from an adjacent island in the array by an opening that fluidly couples the first nanofluidic channel to the second nanofluidic channel, the first nanofluidic channel,
   the second nanofluidic channel, and the islands are arranged so that a fluidic resistance of the first nanofluidic channel increases relative to a fluidic resistance of the second nanofluidic channel along a longitudinal direction of the first nanofluidic channel such that, during use of the nanofluidic device, a portion of a fluid sample flowing through the first nanofluidic channel passes through one or more of the openings between adjacent islands into the second nanofluidic channel,
a width of the first nanofluidic channel repeatedly alternates between a narrow region and an enlarged region along the longitudinal direction of the first nanofluidic channel, and
for each island, a contour of a first side of the island substantially matches a contour of the first undulating outer wall of the first channel facing the first side of the island such that a width between the first side of the island and a boundary of the first undulating outer wall is constant.
17. The method of any one of sentences 1-4, wherein the nanofluidizer may include:
   a first nanofluidic channel;
   a second nanofluidic channel extending along the first nanofluidic channel; and
   a first array of islands separating the first nanofluidic channel from the second nanofluidic channel, wherein a boundary of the first nanofluidic channel is defined by a first undulating outer wall,
wherein each island is separated from an adjacent island in the array by an opening that fluidly couples the first nanofluidic channel to the second nanofluidic channel,
the first nanofluidic channel, the second nanofluidic channel, and the islands are arranged so that a fluidic resistance of the first nanofluidic channel increases relative to a fluidic resistance of the second nanofluidic channel along a longitudinal direction of the first nanofluidic channel such that, during use of the nanofluidic device, a portion of a fluid sample flowing through the first nanofluidic channel passes through one or more of the openings between adjacent islands into the second nanofluidic channel,
a width of the first nanofluidic channel repeatedly alternates between a narrow region and an enlarged region along the longitudinal direction of the first nanofluidic channel, and
for each island, a contour of a first side of the island substantially matches a contour of the first undulating outer wall of the first channel facing the first side of the island, and a radii of curvature of the first undulating outer wall through a first turn of the first nanofluidic channel is smaller than a radii of curvature of the first undulating outer wall through a second adjacent turn of the first nanofluidic channel
18. The method of any one of sentences 1-4, wherein the nanofluidizer may include:
an impeller mounted on a rotatable shaft, a power source to rotate said shaft and impeller, said shaft and impeller being located in a channel having an input and output, said channel containing a support and one or more screens,
each said screen comprising a tapered apertured wall formed into a frusto-conical shape, with a wide end of each said screen being open and a narrow end being at least partially closed, each said screen having a substantially cylindrical section, said section surrounding and extending from said tapered apertured wall, a circular flange surrounding and extending radially outwardly from said cylindrical section at an end opposite to said tapered wall, each said screen being designed to be located in said channel with said flange held rigidly within said support so that any particles passing from said input to each output pass through each said screen, each said impeller being shaped and mounted so that a gap between an edge of the impeller and a tapered wall of each said screen mounted in each said support can be maintained substantially constant when said impeller rotates relative to each said screen, and each said screen being independently removable from said machine and replaceable with another said screen, said another screen having a same outside diameter at a wide end and a same inside depth as each other said screen despite variations between the screens to accommodate a height of the cylindrical section, apertured wall thickness and size and shape of apertures, said gap being maintained at a constant value, if desired, without any adjustments being made to the impeller.
19. The method of any one of sentences 1-4, wherein the nanofluidizer may include:
an impeller mounted on a rotatable shaft,
a power source to rotate said shaft and impeller, said shaft and impeller being located in a channel having an input and an output, and
a screen having a tapered apertured wall formed into a frusto-conical shape with a wide end of said screen being open and a narrow end of said screen being at least partially closed, said screen having a substantially cylindrical section, a height of said cylindrical section being determined by a wall thickness of the screen, and a circular flange surrounding and extending outwardly from said wide end of said screen, and
said channel containing a support, said screen being held within said support so that any particles passing from said input to said output pass through said screen, said impeller being shaped and mounted so that a gap between an edge of said impeller and the tapered wall of said screen remains substantially constant as said impeller rotates relative to said screen, said screen being removable from and replaceable in said support with a replacement screen, a height of the cylindrical section of the replacement screen varying with wall thickness relative to the other screen so that an outside diameter at the wide end and an inside depth of the two screens can be maintained substantially constant.
20. The method of any one of sentences 1-4, wherein the nanofluidizer may include:
an impeller associated with an impeller drive shaft and a drive mechanism to rotate the impeller drive shaft wherein the screen holder comprises;
a. a first flange having a top surface, a bottom surface and an opening wherein the first flange bottom surface includes a screen pilot;
b. a second flange having a second opening; and
c. at least one support arm uniting the first flange with the second flange.
21. The method of any one of sentences 1-4, wherein the nanofluidizer may include:
an impeller associated with an impeller drive shaft and a drive mechanism to rotate the impeller drive shaft wherein the screen holder comprises:
a. a first flange having a top surface, a bottom surface and including a circular opening wherein the first flange bottom surface includes a screen pilot that comprises a circular groove including a first surface and a second surface that together define an angle of from 80 to about 100 degrees;
b. a second flange having a second circular opening wherein the second flange circular opening is smaller in diameter than the first flange circular opening; and
c. at least one support arm uniting the first flange with the second flange.
22. The method of any one of sentences 1-4, wherein the nanofluidizer may include:
an impeller, an impeller drive shaft, a frustoconical screen, and a drive mechanism wherein the adjustable impeller comprises:
a. an impeller having at least one arm and a hub including central aperture wherein the central aperture includes a threaded portion; and
b. an impeller drive shaft associated with the drive mechanism and having a first end and a second end associated with the drive housing wherein the impeller drive shaft includes a threaded portion that is complementary to the impeller central aperture threaded portion and wherein the impeller drive shaft first end includes an impeller to impeller drive shaft lock.
23. The method of any one of sentences 1-4, wherein the homogenizer may be a Quadro HV emulsifier and Wet Mill, carried out at a speed of about 70 m/s.
24. The method of any one of sentences 1-4, wherein the nanofluidizer may include:
mixing the formulation to be pulverized with high pressure working gas into a gas-solids suspension,
conveying of the gas-solids suspension through acceleration nozzles to a pulverizing chamber of a counterjet pulverizer for autogenic pulverizing
conveying the pulverized gas-solids suspension to an intermediate tank, said steps of mixing, conveying to a pulverizing chamber and conveying to an intermediate tank comprising a pulverizing cycle,
removing the gas from the gas-solids suspension and collecting the solids in the intermediate tank,
returning solids from the intermediate tank to be pulverized together with new raw material till there is a desired circulation load, and
continuing the process so that as much end product material is removed from the process as raw material is added to the process, and
wherein said mixing step includes the step of
   feeding the formulation alternately into side by side double-valve feeders and balancing tanks, where a feed pressure in each double-valve feeder is high than a regular feed pressure in an associated balancing tank, and
   synchronizing the double-valve feeders so that an after pressure left in one double valve feeder after release of the formulation into the associated balancing tank is utilized as initial pressure in the other double-valve feeder after receipt of materials therein.
25. The method of any one of sentences 1-24, wherein the step of enriching the formulation may include a step selected from:
a) mixing the one or more formulations with revitalized water;
b) mixing the one or more formulations with a water soluble polyalkylene oxide derivative of a partial long chain fatty acid ester of a compound selected from the group consisting of polyhydric alcohols and their anhydrides, and the said agent having a melting point below 100° C.;
c) subjecting the one or more formulations to photonic energy enrichment; and
d) subjecting the one or more formulations to laser enrichment.
e) infusing Hydrogen (H₂) Gas into the one or more formulations.
26. The method of sentence 25, wherein the step of enriching may include mixing the formulation with revitalized water.
27. The method of sentence 26, wherein the revitalized water may be prepared by inducing water vortices to a natural water input and the formulation is mixed with the revitalized water to achieve an enriched formulation.
28. The method of sentence 25, wherein the one or more formulations may be mixed with a water soluble polyalkylene oxide derivative of a partial long chain fatty acid ester of a compound selected from the group consisting of polyhydric alcohols and their anhydrides, wherein the the water soluble polyalkylene oxide may optionally be a polyethylene oxide derivative of a partial ester of sorbitan with a higher fatty acid.
29. The method of sentence 25, wherein the step of enriching may include subjecting the one or more formulations to photonic energy comprising placing a source of photonic energy to a location proximate to the one or more formulations thereby emitting subatomic photonic light energy to the one or more formulations.
30. The method of sentence 25, wherein the laser enrichment may include subjecting the formulation to an apparatus comprising a laser producing a collimated or near collimated beam, a phase cancellation optical element through which said beam is passed, said phase cancellation optical element being formed by a series combination of a first diffraction grating, a refractive element and a second diffraction grating, whereby a pattern of interference of constructive and destructive nodes is formed in which a diameter of the beam is set to be a sufficiently low multiple of a wavelength of a beat frequency to allow a substantial Fresnel zone to be apparent in the beam.
31. The method of sentence 25, wherein the laser enrichment may include subjecting the formulation to an apparatus comprising a laser of multiple line cavity resonance consisting of a laser diode with a collimated or near collimated beam, said beam being passed through a phase cancellation optical element having a characteristic of canceling several central lines of the laser frequency while leaving higher and lower frequencies generally uncanceled such that a beat frequency of passed frequencies forms a pattern of interference of constructive and destructive nodes in which a diameter of the beam is set to be a sufficiently low multiple of a wavelength of the beat frequency to allow a substantial Fresnel zone to be apparent in the beam and in which an aperture is provided to select a portion of the Fresnel zone wherein a substantial majority of the destructive nodes are apparent relative to the constructive nodes and in which means are provided to modulate the laser frequency.
32. The method of sentence 25, wherein the laser enrichment may include subjecting the formulation to a step of stimulation of molecular resonance by the application of very low intensity electromagnetic radiation modulated at resonant frequencies of molecules of high Q by use of a laser of multiple line cavity resonance consisting of a laser diode with a collimated or near collimated beam, said beam being passed through a phase cancellation optical element said cancellation element having a characteristic of canceling several central lines of a laser frequency while leaving higher and lower frequencies generally uncanceled such that a beat frequency of passed frequencies forms a pattern of interference of constructive and destructive nodes, in which method a diameter of the beam is set to be a sufficiently low multiple of a wavelength of the beat frequency to allow a substantial Fresnel zone to be apparent in the beam and in which an aperture is provided to select a portion of the Fresnel zone wherein a substantial majority of the destructive nodes are apparent relative to the constructive nodes and in which means are provided to modulate the laser frequency.
33. The method of sentence 25, wherein the laser enrichment may include a step of subjecting the formulation to laser radiation from at least two different lasers, wherein the laser radiation has pulses with an effective average pulse length of no more than about 10⁻⁹ seconds, and the laser radiation from each said laser has a different wavelength.
34. The method of sentence 25, wherein the laser enrichment may include a step of subjecting the formulation to laser radiation through a Strachan Device, the Strachan Device comprising a first diffraction grating, a second diffraction grating and a refractive element positioned between the first and second diffraction gratings, canceling a portion of the laser radiation by destructive interference, and producing effective pulses of the laser radiation by constructive interference.
35. The method of sentence 25, wherein the laser enrichment may include a step of subjecting the formulation to laser radiation to improve the bioavailability of the one or more biologically active agents, wherein the laser radiation modifies the one or more biologically active agents to thereby modify reactions within the subject.
36. The method of sentence 35, wherein the laser radiation may provide one or more of the following benefits: reducing inflammation associated with autoimmune diseases, facilitates modification of reaction by-products in the subject, increasing homogenization and flattening of the molecular shape of the formulation, and improving methylation.
37. The method of sentence 35, wherein the enrichment may include infusing H2 gas infused water into the one or more formulations wherein the pH of the formulation is not impacted.
38. In a second aspect, the present invention relates to methods of delivering the one or more formulations prepared by the method of any one of sentences 1-37, comprising contacting said one or more formulations with a subject, wherein said one or more active ingredient(s) is absorbed by said subject.
39. The method of sentence 38, wherein in the contacting step, the one or more formulations may be contacted with an oral mucosa of the subject, whereby said one or more active ingredient(s) is absorbed into a bloodstream of said subject.
40. The method of sentence 38, wherein in the contacting step, the one or more formulations may be applied to skin of the subject, whereby said one or more active ingredient(s) is absorbed into a bloodstream of said subject.
41. The method of sentence 38, may further comprise steps of adding the one or more formulations to a skin-interface membrane of a transdermal drug delivery device configured to be coupled to the skin of the subject, in the contacting step, positioning the transdermal drug delivery device with the skin-interface membrane coupled to the skin of the subject, and optionally repeating the adding and contacting steps to modulate and control dosing to provide blood plasma concentration peaks and troughs in the subject over an extended time period.
42. The method of sentence 38, wherein the subject may be a plant, and the one or more formulations is delivered to a root system of the plant to provide one or more of enhanced nutrient absorption and water uptake, efficient nutrient absorption, enhanced root growth, and a reduction of volume and concentration of nutrients required for delivery to said plant.
43. The method of sentence 38, wherein the subject may be located in an aqueous environment and the one or more formulations is delivered into the aqueous medium to provide one or more of enhanced nutrient absorption, efficient nutrient absorption, enhanced growth of the organism, and a reduction of volume and concentration of nutrients required for delivery to said organism.
44. The method of any one of sentences 38-43, wherein the one or more formulations may be delivered to the subject as a spray.
45. The method of any one of sentences 38-43, wherein the one or more formulations may be delivered to the subject via a protein carrier.
46. In a second aspect, the present invention relates to formulations prepared by the method of any one of sentences 1-45.
47. The formulation of sentence 46, wherein the one or more biologically active agents may have an average particle diameter of from about 1000 nm to about 1 nm or below, as measured by photon correlation spectroscopy.
48. The formulation of any one of sentences-46-47, wherein the one or more biologically active agents may be selected from the group consisting of beet root powder, cannabinoids, essential oils, vitamin A, vitamin D3, vitamin E, coenzyme Q10, cyclopropyl-N-{2-[(1S)-1-(3-ethoxy-4-methoxyphenyl)-2-(methylsulfonyl)ethyl]-3-oxoisoindoline-4-yl}carboxamide, (+)-{2-[1-(3-ethoxy-4-methoxyphenyl)-2-methylsulfonylethyl]-4-acetylaminoisoindoline-1,3-dione, methycobalamin, L-arginine, L-citrulline, L-glutamine, L-lysine, L-ornithine, glycine, L-tyrosine, L-leucine, L-isoleucine, L-valine, L-Theanine, 5-HTP, para amino benzoic acid, gamma amino butaric acid, sodium nitrite, pine bark, melatonin, adenosine, jujube, garcinia gambogia, hoodia gordoni, piperine, green tea, blue cohosh root, burdock root, echinacea root, ginkgo biloba leaf hops, magnolia bark, propolis, skull cap, slippery elm bark, valerian root, wood betony, yucca, Super B-Complex, vitamin C, cholecalciferol, d-alpha-tocopherol, nicotinamide, niacinamide, niacin, pantothenic acid,, riboflavin, pyridoxine, thiamine, folic acid, biotin, cyanocobalamin, inositol, citicoline, L-ascorbic acid, zinc, zinc gluconate, potassium, sodium, chlorine, calcium, phosphorus, iodine, molybdenum, selenium, magnesium, manganese, cobalt, bromine, nickel, boron, silicon, vanadium, chromium, iron, silver, copper, lithium, aluminum, strontium, germanium, lead, rubidium, tin, aspirin, ibuprofen, human growth hormone, ezetimibe, simvastatin, atorvastatin free acid, atorvastatin calcium, and rosuvastatin calcium.
49. The formulation of any one of sentences 46-48, may further include one or more flavoring agents or sweeteners selected from the group consisting of blueberry flavor, orange flavor, berry flavor,, grape flavor, apple flavor, pear flavor, orange cream flavor, mango flavor, passion fruit flavor, tropical flavor, lemon flavor, tangerine flavor, strawberry flavor, pomegranate flavor watermelon, other fruit flavors, spearmint flavor, menthol flavor, coconut flavor, chocolate flavor, vanilla flavor, cinnamon flavor, cream flavor, cookie flavors, candy flavors, meat flavors, xylitol, agave nectar, honey, stevia and other sweeteners
50. The formulation of any one of sentences 46-49, may include the following biologically active agents selected from any one of Tables A-C:

TABLE A

| | Dosing Ranges in mg | |
|---|---|---|
| Biologically active agents | Minimum | Maximum |
| Vitamin B12 (cyanocobalamin or | .12 | 30 |
| Methycobalamin)) | | |

TABLE B

| | Dosing Ranges in mg | |
|---|---|---|
| Biologically active agents | Minimum | Maximum |
| Vitamin B1 (Thiamine) | 0.12 | 6.0 |
| Vitamin B2 (Riboflavin) | 0.13 | 6.5 |
| Vitamin B3 (Niacinamide) | 1.6 | 80.0 |
| Vitamin B5 (Pantothenic | 0.5 | 25.0 |
| Acid) | | |
| Vitamin B6 Pyridoxine | 0.17 | 8.5 |
| Vitamin B7 (Biotin) | 0.03 | 1.5 |
| Vitamin B9 (Folic Acid) | 0.04 | 2.0 |
| Vitamin B12 | 0.001 | 25 |
| (Cyanocobalamin or | | |
| methylcobalamin | | |

TABLE C

| | Dosing Ranges in mg | |
|---|---|---|
| Active Ingredients | Minimum | Maximum |
| CoQ10 (Ubiquinone or Ubiquinol) | 2.5 | 137.5 |
| Vitamin E (Tocophersolan) | 3.9 | 214.5 |
| L-Carnitine | 5 | 275 |

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objectives and features thereof.

### BRIEF DESCRIPTION OF THE DRAWINGS

FIG. 1A is a graphic comparison of mucosal absorption of nanofluidized B-12 versus gastro-intestinal absorption via oral administration of a commercially available B-12 in tablet form and is indicative of the percent change in circulating B-12 concentration as a function of time.

FIG. 1B is a graphic comparison of mucosal absorption of nanofluidized B-12 versus gastro-intestinal absorption via oral administration of a commercially available B-12 in tablet form and is indicative of the relative increase in circulating B-12 concentration in picograms/mL as a function of time.

FIG. 2 is a summary of initial hematological results obtained from a whole blood sample from a 65-year-old male patient, suffering from pernicious anemia induced by vitamin B-12 deficiency, prior to the sublingual administration of nanofluidized vitamin B-12 delivered via spray according to the present invention.

FIG. 3 is a photograph of the sample analyzed in FIG. 2 on a test slide under a high-powered microscope, illustrating erythrocyte abnormalities (anisocytosis and ovalocytes).

FIG. 4 is a summary of initial hematological results obtained from a whole blood sample from a 37-year-old female patient, suffering from pernicious anemia induced by vitamin B-12 deficiency, prior to the sublingual administration of nanofluidized vitamin B-12 delivered via spray according to the present invention.

FIG. 5 is a photograph of the sample analyzed in FIG. 4 on a test slide under a high-powered microscope, illustrating an erythrocyte abnormality (macrocytosis).

FIG. 6 is a summary of initial hematological results obtained from a whole blood sample from a 57-year-old male patient, suffering from vitamin B-12 deficiency, prior to the sublingual administration of nanofluidized vitamin B-12 delivered via spray according to the present invention.

FIG. 7 is a summary of final hematological results obtained from a whole blood sample from the 65-year-old male patient in FIGS. 2 and 3, subsequent to the sublingual administration of nanofluidized vitamin B-12 delivered via spray according to the present invention for a 30-day test period.

FIG. 8 is a photograph of the sample analyzed in FIG. 7 on a test slide under a high-powered microscope, illustrating normal erythrocyte size and shape.

FIG. 9 is a summary of final hematological results obtained from a sample from the 37-year-old female patient in FIGS. 4 and 5, subsequent to the sublingual administration of nanofluidized vitamin B-12 for a 30-day test period.

FIG. 10 is a photograph of the microscope slide of the sample analyzed in FIG. 9 on a test slide under a high-powered microscope, illustrating normal erythrocyte size and shape.

FIG. 11 is a summary of final hematological results obtained from a sample from the 57-year-old male patient in FIG. 6, subsequent to the sublingual administration of nanofluidized vitamin B-12 for a 30-day test period.

FIG. 12 is a schematic and chart demonstrating how the body mechanically breaks down particles. As shown in the schematic and chart, chewing digestive enzymes only break down biopolymers, not aggregates, whereas methods of the present invention use biophysics to digest aggregates into molecular dispersions, suspensions, and emulsions that are optimized for cellular absorption and biological activity.

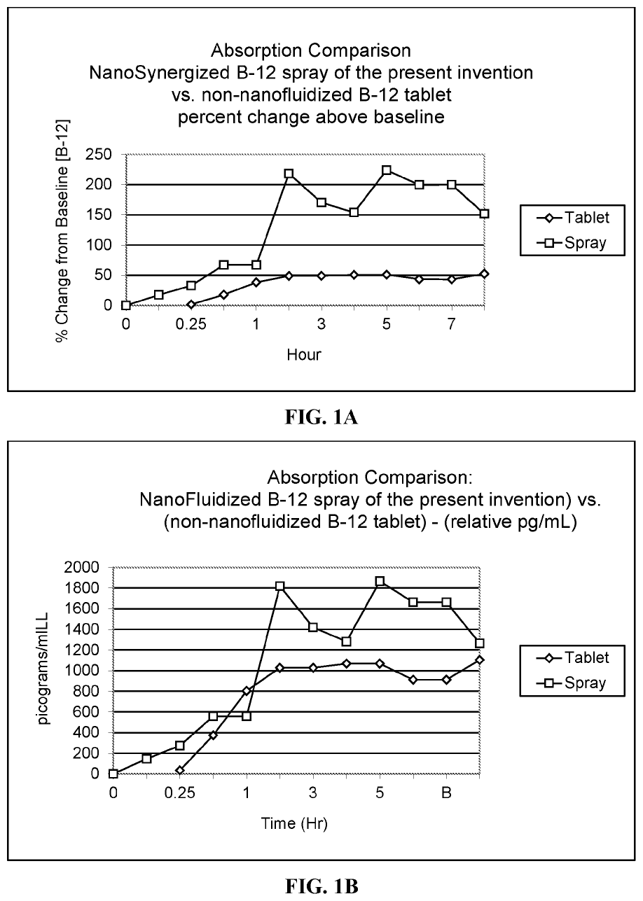

### DETAILED DESCRIPTION OF THE INVENTION

The present invention relates to methods for the production of stable formulations comprising molecular dispersions containing water soluble, water-insoluble or only slightly soluble, at room temperature, solid biologically active agents. The particles have an average particle diameter from 1 nm-1000 nm. Thus, a low microparticle content is allowed in the particle population.

An advantage of the present invention is the ability to form stable formulations with low surfactant concentrations or without surfactants. Thus, preferably the formulations include no surfactants or stabilizers and if used will use no more than 5% surfactants.

A dispersed phase may be a solid in liquid or solid in a semi-solid, whereby the dispersed phase includes one or more active agents that may optionally be pure or substantially pure. The average particle diameter of the dispersed phase may be between 1 nm and 1000 nm (determined with photon correlation spectroscopy), preferably with a narrow population distribution, i.e. a narrow population size ranges from two times as large to one half as small as the median particle size.

### Advantages of the Formulations

The use of the present formulations for drug delivery provides many advantages from pharmaceutical-technological, biopharmaceutical, pharmacological and medical points of view. A few examples are:
The uptake rate increases with an increase in the particle surface according to the law of Noyes-Whitney. This corresponds to an increase in the flow rate of active ingredients in drug delivery whereby the maximum plasma level is achieved faster (e.g. oral or intravenous application of a formulation). The present formulations are therefore of interest for active agents for which the rate of dissolution is an important factor for bioavailability.
Intravenous delivery of water insoluble active ingredients can be made possible by use of the present formulations. Many active ingredients used in pharmaceuticals are partially insoluble or insoluble in water and/or organic solvents. Formulations processed using application of the current invention may be deliverable orally as liquids avoiding the need, risks and difficulties of intravenous and intramuscular application. Furthermore, liquid oral applications of these nanofluidized dispersions may present a more effective means of delivery to the body as compared with intramuscular injections. Pharmacological testing is generally not possible after oral or intravenous application due to the low bioavailability of such solids as a result of their low solubility. Furthermore, it is typically not possible to prepare a solution of these insoluble active materials having sufficient active agent therein to be suitable for intravenous injection. When a solid active ingredient is administered in the present formulations, it can be injected without blocking blood capillaries. When a nanofluidized formulation including an active ingredient is injected, the active ingredient can dissolve into the blood volume, which is relatively large compared to the injection volume (for example 20 mL to 6 L), the blood proteins often having an additional solubilizing effect.
A reduction in the injection volume of active agents may be achieved by preparing a nanofluidized formulation. When active ingredients with low water solubility are formulated in a solution, a relatively large volume of the solution is required to be administered to a subject to achieve the desired result. Alternatively, when the active ingredient is formulated as a nanofluidized formulation, the same amount of the drug may be dispersed in a saturated solution of the active ingredient with a significantly smaller volume. For example, an infusion could be replaced by a bolus injection.
Nanofluidized formulations may be used for controlled active agent application. After oral administration, oral immunization could take place via the M cells in the gastrointestinal tract, and selective enrichment in absorption windows of the gastrointestinal tract can be achieved with bioadhesives. The formulations of the invention can be coadministered through the use of bioadhesives.
Nanofluidized formulations may be used as delivery systems for targeting of the active agent. After intravenous injection, particles accumulate in certain organs, e.g. liver, spleen or bone marrow, depending on their surface properties (RH Müller, Colloidal Carriers for Controlled Drug Delivery and Targeting, Wissenschaftliche Verlagsgesellschaft, Stuttgart, 1991). After parenteral administration, accumulation in the lymphatic system may occur. Targeted enrichment of the active agent at the site of action can reduce side effects and increase the therapeutic efficiency and thus improve the therapeutic index.

In one aspect, this application is directed towards application of nanofluidized formulations for delivery of biologically active agents, either singly or in various combinations, e.g. multi-vitamin/mineral supplements. As an illustrative, albeit non-limiting example, the Figures show that fat-soluble Coenzyme Q10 (CoQ10), when administered via the mucosal membrane as a spray, achieves higher blood concentration of CoQ10 when administered as a nanofluidized formulation, as compared to CoQ10 administered in a liquid formulation that was not nanofluidized. By extension, this aspect applies to other insoluble or partially soluble biologically active agents that can be provided in nanofluidized formulations.

While not wishing to be bound to any theory of operation, there are several mechanisms that may account for the increased absorption of CoQ10, or other biologically active agents, when formulated as a nanofluidized formulation via the oral mucosal route. First, it is theorized that there is a greater concentration of biologically active agent at the active mucosal surface. There are two possible explanations for this phenomenon:
1) Increased Concentration at the Mucosal Membrane—Using the nanofluidized formulations, more molecules can be dispersed in a smaller unit volume of fluid thereby allowing a greater number of molecules to come into contact with the mucosal membrane, over a shorter period of time. This increases the absorption of the biologically active agent via the mucosal membrane and enhances the probability that more molecules will be absorbed than from non-nanofluidized formulations.
2) Increased Passive Infusion—As a result of the increased local concentration of the biologically active agent, there may be a greater passive diffusion gradient across the mucosal membrane, ultimately resulting in greater plasma levels.

Another theory is nanofluidized formulations stimulate active transport of the molecules across the mucosal membrane. It is theorized that the nanofluidized formulations may stimulate greater “active transport” of compounds across the mucosal membrane by bringing a greater concentration of active agent into contact with specific receptor sites.

In another aspect, the present invention also provides a method for the delivery of a biologically active agent enhanced by the formation of a stable uniform formulations. While illustrative examples are limited to human subjects, the technology is in no way limited by said examples. The formulations which are the subject of the instant invention are contemplated for use in medical or veterinary settings, or for the delivery of vitamins, minerals, amino acids or other nutrients to plants, animals or humans, and may be administered in any suitable manner known in the art. The preferred embodiment, as illustrated herein, is formulated to be sprayed into the mouth of a human or animal, whereby absorption via the oral mucosa is accomplished. For plants or aquaculture enrichment, the processed formulations can be applied directly into hydroponic water or irrigation water supplies. For aquaculture the processed formulations can likewise be added directly into the water environment or otherwise infused into solid aqua food sources.

A “biologically active agent”, “biological agent”, “active ingredient”, “agent” or “drug”, as used herein, refers to any synthetic or natural element or compound, protein, cell, or tissue including a pharmaceutical, therapeutic, nutritional supplement, herb, hormone, or the like, or any combinations thereof, which when introduced into the body causes a desired biological response, such as altering body function or altering cosmetic appearance.

The terms “vitamin B-12” and “B-12” are used interchangeably herein and refer to any supplemental form known to the skilled artisan including, albeit not limited to: cyanocobalamin, methylcobalamin, adenosylcobalamin, conjugates, mixtures or combinations thereof.

To convert the nanofluidizable mixture to the stable, uniform formulation of the present invention, the mixture may be subjected to treatment with an ultra-high energy mixing device. This is preferably achieved through the process of nanofluidization. A nanofluidizer high pressure homogenizer processor is a device that provides high shear rates, thereby providing a high energy-per-unit fluid volume to produce uniform submicron particles of chemical or particulate substances. Process pressures are highly variable, ranging from about 5,000 psi to about 45,000 psi, enabling the processing of a wide variety of fluids ranging from simple oil-in-water emulsions to high-weight-percent solids-in-liquid suspensions.

The nanofluidizer contains an air-powered intensifier pump designed to supply the desired pressure at a constant rate to the product stream. As the pump travels through its pressure stroke, it drives the product at a constant pressure through precisely defined fixed-geometry microchannels within the interaction chamber. As a result, the product stream accelerates to high velocities, creating shear rates within the product stream that are orders of magnitude greater than other conventional mixers. All of the product experiences essentially identical processing conditions, producing the desired results, including substantially uniform particle size reduction.

As a result of the high shear rate there is produced a mixture containing substantially uniform submicron particles and the creation of stable emulsions and dispersions can be achieved. This processing overcomes limitations of conventional processing technologies by utilizing high pressure streams that collide at ultra-high velocities in precisely defined microchannels. The final product is a stable uniform nanofluidized formulation.

The high pressure homogenizers preferred by the present method provide a combination of high shear, cavitation, and impact in addition to the following features:
a) Enhanced quality controls,
b) Production volumes of 1000 liters of liquid formulation processed an hour at 45,000 psi.
c) Modular and flexible mechanical operation from a non-fixed platform enabling multiple processing set ups.
d) Higher and more constant processing pressures enable reduced variation in process resulting in tighter molecular distribution.
e) Tight particle size distribution of 200 nm to 1 nm
f) Fewer passes required reducing material processing time and machine wear
g) the ability to monitor and record data specific for each product lot enabling product controls and the ability to monitor processing elements for enhancement and optimization of product capabilities. Examples of such data elements may include, flow rates, pressures applied, cleaning cycles, product run times, and other elements that can impact the optimization of the products in processing,
h) real time and remote monitoring of production processes enhancing product consistency to reduce risks of product variability leasing to greater product optimization through product consistency,
i) enable standardization of processing, monitoring, and possibly, integration across multiple production machines or locations, enhancing product consistency and quality, and
j) enable improved regulatory record keeping enhancing quality.

These homogenizers also have the ability to modularize and tailor machine configuration to customize to a desired sizing, geometry, and morphology of small particle sized formulations, enabling a targeted metabolic effect, enrichment and enhanced processing of the formulations. The homogenizer may include a modular intensifier design which enables constant high pressure across stroke cycles. The homogenizers may include heavy duty, long cycle strokes which reduce machine wear and resulting costs.

The homogenizers employed by the present method may be configured to independently control each of shear, cavitation, and impact. This modularity allows the ability to customize each of these three factors to improve results for various applications.

For example, cavitation may be tuned to use gentler or harsher forces by varying nozzle size or operating pressure and back pressure, thereby enabling customizable processing of the formulations. The flow rate may be adjusted to adjust for turbulent premixing vs laminar flow. The shear processing time can be adjusted to be shorter or longer. The homogenization may be carried out at a constant pressure which increases the longevity of seals while eliminating purging and priming issues. This reduces cost.

These homogenizers are particularly useful as they provide the following benefits: A) particle size reduction; B) extension of product shelf life; C) improved sensorial properties such as rapid penetration and merging textures; D) improved biophysical properties such as hydrating power; E) color and flavor retention; F) improved texture; G) supports the growing demand for organic and preservative free products; H) vitamins and antioxidants stay in the formulations rather than being removed and then re-added; and I) pharmaceutical and supplement suspensions that mask the bitter taste of active drugs or supplemental inputs. Improving taste and reducing bitterness will increase user compliance and therefore enhance the beneficial effects of the formulations; J) achievable small, uniform particles, enabling purification of formulations simply by running them through a 0.22-micron filter; K) achieving the desired particle sizes of 1 nm to 1000 nm; L) creating stable emulsions, and dispersions with particles at equilibrium; and M) increased efficacy of the resulting products as described elsewhere herein.

The use of this homogenizer may be applied to provide a more consistent dosing per unit volume thereby reducing the bitter taste of active drugs or supplemental inputs. Such a means of improving taste and reducing bitterness can increase user compliance and therefore the beneficial effects of the formulations.

The stability and rate of absorption may be further enhanced by one or more components within the formulation. In addition, the rate of absorption of the final product may be enhanced by the uniformity or size of the biologically active agent containing particles in the formulations.

Permeation enhancers that may be utilized in the present invention include the conventional physiologically acceptable compounds generally recognized as safe (GRAS) for human consumption. Without being bound to theory, the formulation of the present invention may not include surfactant to assist in decreasing particle size. However, surfactants may be included based on formulation requirements, the surfactant levels will be less than 5 wt. %, based on the total weight of the formulation.

Theoretically, such dispersions should allow molecules to be delivered across tissue barriers at a more even rate than non-nanofluidized or “normal” solutions. Smaller, more uniform size molecules should enable quicker cellular absorption. The smaller molecules absorb into the cell more quickly reducing residual actives remaining in the blood than with molecules prepared by standard pharmacological methods and delivered either by oral mucosal or intestinal absorption.

By using the nanofluidization process to prepare mixtures of biologically active agents, e.g. vitamins and other nutritional supplements, products may be designed, manufactured and standardized for use in spray applicators which deliver single dose sprays to the mucosal membranes. The purpose of this type of delivery is to introduce such biologically active agents, e.g. vitamins and minerals, into the body in a manner which allows, over time, more rapid, uniform and complete absorption than in the form of pills, tablets, capsules or liquids which are absorbed through the gastrointestinal tract. The spray delivery system can likewise reduce the need, risks and inconvenience of less efficient intramuscular injection delivery systems that displace actives in the muscle reducing actives available at the sites of action. In addition, the nanofluidization process appears to offer increases in shelf-life, with testing showing a shelf-life of about 2 years.

In another aspect, a Quadro HV emulsifier and Wet Mill may be used to achieve high shear rotor/stator mixing to pretreat the desired formulations. This homogenizer is designed for emulsification in a single pass, and capable of wet milling some active pharmaceutical ingredients (API's) to achieve particle diameters of about 10-20 microns. Operating at extremely high rotational velocity, tooling tip speeds up to 70 m/s can be achieved providing more than 55 times the shear energy input compared with conventional rotor-stator systems. This performance fills the gap between existing rotor/stator technologies and high pressure homogenizers. It provides dry powder deagglomeration and size reduction, sizing and grading of powders, wet mass granulation for efficient and even drying, fine particle size reduction, size reduction of heat sensitive products, and security screening of free flowing and sticky products.

The present invention may employ the homogenizers of U.S. Pat. No. 4,768,722 to prepare the formulation. Specifically, the homogenizer may include a series of screens that can be used interchangeably with a size reduction machine, a gap size remaining constant without any adjustment, said screens having different wall thicknesses. The size reduction machine may include a mechanism externally of the machine to adjust the size of the gap. The machine may also include a mechanism for setting up the gap between the impeller and the screen of a size reduction machine. The machine may be easily disassembled for cleaning.

The present invention may employ the homogenizers of U.S. Pat. No. 8,651,230 to prepare the formulation. These homogenizers apply sonication through high shear forces created by ultrasonic cavitation to break up particle agglomerates resulting in smaller and more uniform particles sizes. The stable, homogenous suspensions produced by ultrasonics are widely used in many industries today. The application of probe sonication and sonicators to the molecular dispersion formulations improves dispersion, reduces deagglomeration and particle size, mitigates particle synthesis and precipitation, as well as improving surface functionalization.

The present invention may employ the homogenizers of U.S. Pat. No. 7,052,172 to prepare the formulations. These homogenizers employ two distinct types of methods commonly used for molecular dispersions. Mixing of the molecular dispersions, can involve agitating or stirring the components utilizing a low-shear process with no significant micron-level particle size reduction in the joined components. Mixing is often used for combining two soluble fluids, dissolving solids into fluids before the supersaturation point, and similar activities. Additionally, homogenizing, can involve disaggregating or emulsifying the components utilizing a high-shear process with significant micron-level particle size reduction of the molecular dispersion. Homogenizing is often used for creating emulsions, reducing agglomerate particles to increase surface area, and similar activities.

The present invention may employ nanofluidizers according to US 2020/0254407. These portable nanofluidic mixer systems can include a blend application to issue blend instructions to a nanofluidic mixer device. The nanofluidic mixer device includes a housing, nanofluidic pumps and valves within the device housing, a nanofluidic dispenser, a nanofluidic mixer chip, and a mix controller. The nanofluidic mixer chip receives and meters nanofluidic amounts of one or more fluids. The mix controller electronically communicates with the blend application to receive blending instructions. The nanofluidic mixer device includes fluid pathways for fluid communication between one or more fluid canisters and the nanofluidic mixer chip, and between the nanofluidic mixer chip and the nanofluidic dispenser. The mix controller controls the nanofluidic pumps and the nanofluidic valves, to control a system pressure within the nanofluidic mixer device, for the delivery of the one or more fluids to the nanofluidic mixer chip, and to dispense a nanofluidic mixture from the nanofluidic dispenser.

The present invention may employ the nanofluidizer of US 2021/0283610 to prepare the formulations. These nanofluidizers are capable of controlling the geometries and dimensions of nanofluidic devices such that one can manipulate not only the position of particles suspended within a fluid sample, but also portions of the fluid itself to enable substantial increases in particle concentration for large quantities of the fluid sample or to filter fluid samples of undesired particles. For example, careful control of the geometries and dimensions of a nanofluidic device can, in certain implementations, be used to alter the concentration of particles within a fluid sample through shifting the particles across fluid streamlines.

The present invention may employ the nanofluidizer of U.S. Pat. No. 7,461,799 to prepare the formulations. These nanofluidizers are capable of pulverizing material which is then combined with high-pressure to form a gas-solids suspension, which through acceleration nozzles is conveyed to the pulverizing chamber of a counterjet pulverizer for autogenous pulverization. This nanofluidizer is suitable for industrial production of dispersed powders, by means of which it is possible to produce for the processing industry necessary powders and coating agents and pigments which are finer than before and which are most economical, effective and solid. These dispersed powders can be applied to the production of the molecular dispersion formulations.

The present invention may employ the nanofluidizers of EP 2 498 753 to prepare the formulations. These nanofluidizers provide methods of preparation of an oral nanosuspension of a poorly soluble biologically active material with improved bioavailability.

Many new chemical entities are too potent, too toxic, or too water-insoluble, making them undesirable candidates. Potency and toxicity are intrinsic to molecular design and are typically best remedied by altering or refining chemical structure. On the other hand, poor solubility typically leads to poor oral bioavailability, fed/fasted variations in bioavailability, cumbersome and inconvenient dosage forms, and may necessitate the use of harsh solubilizing agents that are associated with adverse side effects. A new generation of nanoparticulate delivery systems specifically designed for resolving formulation issues associated with these poorly water-soluble compounds can be used to address one or more of these problems (see, e.g., Liversidge G, Cundy K. “Particle size reduction for improvement of oral bioavailability of hydrophobic drugs: I. Absolute oral bioavailability of nanocrystalline danazol in beagle dogs”, Int. J. Pharm. 1995, 125:91-97).

Molecular dispersions, in comparison to micronized drug particles, have significantly greater surface area. The increase in surface area enhances dissolution rate, thereby improving delivery efficiency for the most commonly-used routes of administration (Jinno J, et al. “Effect of particle size reduction on dissolution and oral absorption of a poorly water-soluble drug, cilostazol, in beagle dogs”, J. Cont. Rel. 2006, 206 (111): 56-64).

The formulations prepared by the method of the present application may deliver biologically active agents in amounts that provide dosing ranges to reduce the risk of toxicity. The present invention also relates to methods and systems for determining suitable dosing ranges for delivering the biologically active agents to a subject. The system includes a computational biology platform for scalable integration of molecular pathway models to enable predictive and quantitative understanding of complex biomolecular processes and diseases to determine risk, toxicity, and efficacy early in the product development process. For example, human cells may be tested for their ability to absorb each biologically active agent. A minimum dosing value is determined when a concentration of the biologically active agent is detected by the testing system. The maximum dosing value is determined when a human cell has achieved full saturation of the biologically active agent. The data for a single cell can be extrapolated for determining a saturation dosage of the biologically active agent. Once full saturation is achieved, any excess biologically active agent would be dispensed out of the body with no additional benefit. Computational testing methods to determine dosing ranges will be developed for each individual active agent. Minimum dosing levels will be determined by the initial entry point of the active agent into the cell, Cellular saturation levels will also be determined and established as a maximum dose unless toxic levels are achieved prior to cellular saturation.

The methods of preparing the formulations of the present invention may be particularly helpful for reducing nutritional deficiencies. Formulations prepared by the method of the present invention may provide improved absorption of biologically active agents. For example, the formulations of a Vitamin B-12, Super B-Complex and CoQ-10 prepared by the methods of the present invention may include the following biologically active agents, formulations and/or characteristics:
1) Vitamin B-12-Vitamin B-12 has been used for treatment of anemia, energy deficiencies, autism, depression, and even for improving female fertility among other conditions. B-12 is not found in plant-based foods, making the world's vegetarian populations, at particular risk of B-12 deficiency. In a clinical study, patients with Pernicious Anemia using nanofluidized Vitamin B12 were able to regenerate their abnormal red blood cells to normal within 30 days. This would take much longer to accomplish even with Vitamin B12 injections. In addition, new findings show that Vitamin B12 assists in keeping the mental capacities sharp in older ages by deferring onset of dementia in the elderly.

For example, a composition including vitamin B-12 may include the following active ingredients in the following dosing ranges (mg):

| | Dosing Ranges in mg | |
|---|---|---|
| Active Ingredients | Minimum | Maximum |
| Vitamin B12 (Cyanocobalamin or | 0.12 | 30 |
| Methycobalamin) | | |

2) Super B-Complex-B vitamins play a vital role in maintaining good health and well-being. As the building blocks of a healthy body, B vitamins have a direct impact on your energy levels, brain function, and cell metabolism. This includes assisting in lowering homocysteine levels in patients and treating serious cases of anemia. B vitamins, including folic acid, B6, and B12, may help lower blood levels of homocysteine. Research has implicated high homocysteine levels in heart disease and stroke.

For example, a composition including Super B-Complex may include the following active ingredients in the following dosing ranges (mg)

| | Dosing Ranges in mg | |
|---|---|---|
| Active Ingredients | Minimum | Maximum |
| Vitamin B1 (Thiamine) | 0.12 | 6.6 |
| Vitamin B2 (Riboflavin) | 0.13 | 7.2 |
| Vitamin B3 (Niacinamide) | 1.6 | 88.0 |
| Vitamin B5 (Pantothenic Acid) | 0.5 | 27.5 |
| Vitamin B6 Pyridoxine | 0.17 | 9.35 |
| Vitamin B7 (Biotin) | 0.003 | .165 |
| Vitamin B9 (Folic Acid) | 0.04 | 2.2 |
| Vitamin B12 (Cyanocobalamin | 0.001 | 25 |
| or Methylcobalamin) | | |

3) CoQ10 Complex—Coenzyme Q10 is a natural antioxidant synthesized in the body. Coenzyme Q10 is vital to just about every function in the body especially involving oxygen utilization and energy production in the heart. It assists in maintaining the normal oxidative state of LDL cholesterol, helps assure circulatory health, and supports optimal functioning of the heart muscle. CoQ10 may also help support the health of vessel walls. Statin Drug (Lipitor, Crestor etc.) users are particularly at risk for CoQ10 deficiency. When CoQ10 is added to a supplementation program, bioavailability absorption are dramatically increased. When compared to the market leading CoQ10 in a comparative 29-day blood study, it was found that the nanofluidized CoQ10 was over 18 times better absorbed at 100 mg or over 4.5 times better absorbed at a 25 mg dose.

For example, a composition including CoQ10 may include the following active ingredients in the following dosing ranges (mg):

| | Dosing Ranges in mg | |
|---|---|---|
| Active Ingredients | Minimum | Maximum |
| CoQ10 (Ubiquinone or Ubiquinol) | 2.5 | 137.5 |
| Vitamin E (tocophersolan) | 3.9 | 214.5 |
| L-Carnitine | 5 | 275 |

Nutrient formulations can be used to grow superior organic fruits and vegetables. This process will yield better quality produce in less time with an increasing nutritional value to the consumer. In this manner, enriched nutrients can be supplied to the root structure of plants increasing the capability of absorption. Recirculating and processed rich nutrient formulations may enhance the absorption, speed of growth and quality of hydroponically grown crops:
Increase in the rates of nutrient absorption and water uptake #Enhanced root growth
Efficient uptake of nutrients
Reduction of volume and concentrations of nutrients
Ratios at which nutrients are absorbed

A “Nutrient Film Technique (NFT)” is defined as a thin film of oxygen rich nutrient formulation that flows gently down sloped, flat-bottomed gullies (plastic channels), which contain the plant roots with no solid planting media in a hydroponic system.

Recirculating and processed nutrient formulations provide a useful and unique system in the stage of plant growth and rates of nutrient uptake by the plant's root mat. Also, a decreased dose of nutrient solution may be all that is required by the crop, resulting in optimum growth.

The present invention also relates to a system for the enrichment and preparation of formulations. The system includes several modules for the processing, enrichment, delivery, formulation, and absorptive measurement that may be combined in different combinations for making formulations of supplements and pharmaceuticals. . . . An example of such a system is described below. The system of the invention may include some or all of the modules set forth herein in any combination.

A first module of the system is employed to identify a suitable dosing range for each biologically active agent employed in the formulation. The first module may be used to implement a process including steps of testing a cell's ability to absorb the one or more water soluble, partially water soluble active ingredient(s) and/or the one or more partially oil soluble active ingredient(s), as determined by using a computational system for scalable integration of molecular pathway models to enable predictive and quantitative understanding of complex biomolecular processes and diseases to determine risk, toxicity, and efficacy. This platform enables product manufacturers to gain insights to understand how particular ingredients and/or single molecules act upon a particular indication or molecular mechanism of action. The detailed insights provide manufacturers a powerful capability to determine how to formulate truly efficacious products and articulate the value of those ingredients and formulations to their customers and consumers.

Examples of suitable computational systems that may be employed in the present invention are taught by U.S. Publication No. 2015/0305630, U.S. Publication No. 2012/0095735; Al-Lazikani, Bissan, et al. “Combinatorial Drug Therapy for Cancer in the Post-Genomic Era.” Nature Biotechnology, vol. 30, no. 7, July 2012, pp. 679-692, https://doi.org/10.1038/nbt.2284; Palsson, Sirus, et al. “The Development of a Fully-Integrated Immune Response Model (FIRM) Simulator of the Immune Response through Integration of Multiple Subset Models.” BMC Systems Biology, vol. 7, no. 1, 2013, p. 95, https://doi.org/10.1186/1752-0509-7-95; Fox, Robert J, et al. “Setting a Research Agenda for Progressive Multiple Sclerosis: The International Collaborative on Progressive MS.” Multiple Sclerosis Journal, vol. 18, no. 11, 23 Aug. 2012, pp. 1534-1540, https://doi.org/10.1177/1352458512458169; Shiva, A, and C. Forbes Dewey. CytoSolve: A Scalable Computational Method for Dynamic Integration of Multiple Molecular Pathway Models. Vol. 4, no. 1, 1 Mar. 2011, pp. 28-45, https://doi.org/10.1007/s12195-010-0143-x; Koo, Andrew B, et al. In Silico Modeling of Shear-Stress-Induced Nitric Oxide Production in Endothelial Cells through Systems Biology. Vol. 104, no. 10, 21 May 2013, pp. 2295-2306, https://doi.org/10.1016/j.bpj.2013.03.052; the computational systems and methods described in the aforementioned references are hereby incorporated by reference in their entirety as if fully set forth herein.

The aforementioned computational systems are advantageous for formulating suitable dosing ranges since they can be used to produce precise workable formulations to deal with problem diseases or preexisting conditions, they can mitigate toxicity by modeling to determine proper dosages, thereby avoiding dosage problems and toxic dosages for single or multi-ingredient vitamins and/or supplements or other biologically active agents, and they can produce optimal dosages for single or multi-ingredient combinations of vitamins, supplements, and/or other biologically active agents.

Traditional methods of target discovery have relied on in vitrolin vivo studies and sometimes molecular dynamics analysis. However, these methods are ad hoc and do not use the computational system biology approach based on a mechanistic understanding of the underlying biology. Target identification is a key step in drug development. Based on systematic literature review in disease, targets are identified from a cell or microenvironment. Differential sensitivity of individual drugs and molecular targets in computational models are validated with in vivo and in vitro experimental data from the literature repository. Finally, validated computational models can be used to avoid repetitive wet lab experiments. Target identification and validation would be concurrently used to test the combination of different drugs. The computational systems disclosed herein and in silico mechanistic models provide a breakthrough methodology to discover and validate targets cheaper and faster.

The present method of using computational models disclosed herein were employed to optimize a combination of curcumin and resveratrol, two natural anti-inflammatory compounds. In silico models of inflammation were developed and individuals tested, also, the combination of curcumin and resveratrol on the levels of inflammation was determined. Although both curcumin and resveratrol reduced the inflammation levels by 66% and 60%, respectively, when combined at much lower dosage levels, the inflammation was reduced by 75%. This is an example of the synergistic effect of combination drugs that can achieve higher efficacy at low dosage levels of therapeutics, employing the method of the present invention.

Diverse responses of target or biomarker induced toxicity through different signaling pathways are identified through systematic literature review. Computational models are constructed to identify the impact of each drug or ingredient on susceptible targets or biomarkers. PancreaSolve's platform evaluates the paradox effect of drugs or ingredients in combination which enables the safety and toxicity analysis. Doses of either drug and/or ingredient in various combinations are tested to identify the threshold dose required to provide a desired effect.

The effect of the combination of arginine and caffeine on nitric oxide production was evaluated. This combination is sometimes found in dietary supplements taken by military personnel. The analysis revealed that arginine alone increased the nitric oxide concentration whereas caffeine alone reduced the nitric oxide concentration, both in healthy individuals. In combination, the results indicated that production rate in healthy individuals did not increase significantly by arginine supplementation. On the other hand, arginine supplementation attenuated a near zero production rate to nearly normal levels in hyperglycemic/hypertensive individuals.

Low impact, minimally intrusive neurological and physiological testing can be applied through the use of Heart Rate Variability monitoring and/or monitoring and/or measurement of electric fields in the body to enable rapid determination of the effects on the brain and the body of altered dosage levels of biologically active agents on the subjects. By applying adjusted levels of the biologically active agent to the subject and measuring HRV, efficient and effective dosing levels and their resulting metabolic effects can be defined.

For example, the method may further comprise a step of testing a subject that received the one or more enriched formulations using a transformative non-contact, optionally artificial intelligence (AI) powered remote photonic virtual health testing, such as taught by U.S. Pub. No. US2022/0254502 A1. The non-contact and AI powered remote photonic virtual health testing may include a non-contact and non-invasive apparatus for monitoring the health characteristics of a user. The apparatus may include one or more cameras and a processor for collecting and analyzing real-time video of the user and for processing at least each frame from the obtained real-time video. One or more facial regions may be processed to extract images based on physiological monitoring model along with photo plethsmography imagining (iPPG) and Optical Coherence Tomography (OCT) variation to obtain at least one result indicative of health characteristics, such as pulse rate, or respiration rate, or blood oxygen levels (Spo2), or blood pressure, or temperature of the user, or concentration of the one or more enriched formulations in the blood stream of the subject. This testing method may be employed to identify suitable dosing ranges for the patient.

Listed below are relevant Publications from “A Collection of Research Papers on Biomedical Engineering” published by Docsun Biomedical Research Institute (ISBN: 978-620-4-73864-2). This publication describes the use of non-invasive technology in the field of medicine to help in solving everyday health problems.

“NON-INVASIVE BLOOD SUGAR LEVEL MEASUREMENT FOR MONITORING HEALTH STATE, Julian Gerald Dcruz, Jan Yeh, Ted Huang. Docsun Biomedical Research Institute, 4F-2, No.267, Lequn 2nd Rd. Zhongshan District, pages 30-49 discuss using methods of non-invasive technology to determine blood glucose levels through transmittance spectroscopy on the nose lobe, the lower regions of the eyes and lower forehead regions. These non-invasive methods employ, for example spectrometry or analysis of other parameters that are related to glucose levels. The non-invasive methods of monitoring the blood glucose levels are based on the concentration of glucose from its thermal, chemical, electrical or optical sensing properties. Other sensed properties can also be used such as thermal conductivity, electric and acoustic impedance and electromagnetic response. This is due to the human body's different physiological responses to changes in glucose levels.

The measurement of glucose levels can be done on a continuous and real time monitoring basis or it can be done by activation of a single measurement on demand which is treated as a substitute sample of that obtained from the existing invasive methods. The final result can be obtained either immediately or with a particular delay.

Glucose measurement systems can employ one or more of thermal, chemical, electrical, impedance, electromagnetic or acoustic properties. The technology that is used to analyze the sensed information may include optical, transdermal, and thermal conductivity as well as autonomic dysfunction (HRV-based), electromagnetic response and nanotechnology.

Optical noninvasive glucose measurement operates on the principle that light that meets biological tissues is reflected, scattered and transmitted in a manner that us proportional to the structure and chemical components of the sample. The following methods may be used in noninvasive detection of glucose levels in the human body:
a) Infrared spectroscopy.
b) Near-infrared (NIR) spectroscopy. The absorption of light by human tissues is closely related to its wavelength. UV light is easily absorbed by proteins and DNA, visible light by hemoglobin, while infrared light is easily absorbed by water. Near-infrared (NIR, 680-2500 nm) and mid-infrared (MIR, 2500-25,000 nm) have more ideal detection spectra since the NIR range can easily penetrate biofluids and soft tissues and it scatters less than UV or visible light. Therefore, sensing and measurement can be achieved by both reflection and transmission. Various wavelengths are analyzed because of the weak absorption of water and the relatively high energy of the signal that is measured depending on the molecular structure and the absorption spectrum ability. Various medical devices use this technique including Dream Beam™, Diasensor™, SugarTrac™ and MedOptix™. For more accurate results, the measurements are taken in the earlobes, fingers, forearms, oral mucosa and lips.

Transdermal noninvasive glucose measurements can also be employed.

The conventional devices that are used for the monitoring of blood glucose mainly use the electrochemical method. This method requires the drawing of a small amount of blood using either the finger-pricking method or use of a thin lancet that is implanted into the skin. The present invention may employ various noninvasive techniques for the measurement of blood glucose levels. Various technologies are discussed and the criteria for their classification, special focus being given to transdermal and optical based techniques that are both common and suitable for use in glucose monitoring.

Another paper that discusses a suitable methodology for use in the present invention is, “NON-INVASIVE METHODS TO DETECT CHOLESTEROL LEVELS AND HOW IT COMPARES TO STANDARD LIPID TEST,” Julian Gerald Dcruz, Paichang Yeh, Ted Huang and Isabel Karungari Makara. Docsun Biomedical Research Institute, 4F-2, No.267, Lequn 2nd Rd. Zhongshan District, Taipei, Taiwan. See pages 50 to 64.

On such technique is hyperspectral imaging. In this method, the total cholesterol levels of the person are estimated from his/her facial skin lesion (Xanthelasma) using hyper spectral image analysis. Using sensors utilizing infrared light absorption cholesterol level detection is done using sensors utilizing infrared light absorption in body tissues, to simplify the measurement of blood cholesterol levels. It uses a device that monitors cholesterol using an infrared sensor with IR LED-940 nm wavelength as a transmitter. A photodiode detector is used with the wavelength range of 400-1100 nm and a microcontroller as the minimum system for controlling the value of the output voltage in the form of digital data and then converted onto total blood cholesterol.

The paper, “Respiratory Rate Importance in Covid-19 Methods and Solutions Than Can Help Curb Spread of Covid-19,” Julian Gerald Dcruz, Jan Yeh, Ted Huang. Docsun Biomedical Research Institute, 4F-2, No.267, Lequn 2nd Rd., Zhongshan District, pages 65-79 describes alternative smart devices for use in remote respiratory rate monitoring. The following devices can be used for the remote respiratory rate monitoring:
a) a camera, e.g. of a smart device can be used to record the respiratory rate from the respiratory-induced chest wall movements or superficial changes in face perfusion of a patient.
b) a microphone of, e.g. a smartphone can be used to record the respiratory rate from the breathing sounds of the patient,
c) an instrumented mattress can be adopted for continuous respiratory rate monitoring by registering the breathing-related chest wall movements of the patient,
d) radio wave or Wi-Fi signal sources and receivers can be used for registering respiratory rate values through the modulation of the transmitted signals by respiratory-related thoracic movements (no body sensors are needed), and
e) A smart garment (e.g., a strap with conductive strain sensors) can be used to record respiratory rate continuously from the respiratory-related periodic changes in chest wall circumference, even during daily activities (e.g., walking).

Some of these methods involve registering a short video capturing the torso area or the face of the patient, the respiratory rate (and other vital signs like heart rate) can then be streamed for data collection. A good example is The Docsun Health Monitoring System, a device used for the prediction of the physiological state of the recipient by uploading or recording a video of 15 seconds with all the facial features visible. A health report Summary is generated based on the prediction of the Physiological state of the recipient, the summary contains information of vital signs (“biomarkers”) temperature, heart rate, spo2 level, breathing rate, and eye coloration readings predicted by the AI system and a detailed summary based on the predicted vital signs stating whether the patient is having an infection or is in a normal state.

This remote, non-contact optical testing will enable rapid, real-time, self-administered test and monitoring feedback from a wide, dispersed, locationally distant group of enriched formula users. These users and/or their health care providers as well as formulators can track the users results from administration of specific active biologically active agents over time after intaking formulas made by the present methods. Populations in remote areas, the infirm, non-mobile or other difficult to access and/or underrepresented health communities globally can participate in simple, minimally intrusive testing and monitoring. This rapid, portable, accessible, economical, and broad virtual visual optical testing and monitoring ability will improve the feedback loop for products produced by the present methods thereby enabling speedy formulation refinement cycles as well as quickly confirming the efficacy of the enriched formulas on their own or compared to traditional or existing commercial products.

A second module of the system may be employed for enriching the formulation and/or one or more components of the formulation. The enriching module may employ laser enrichment, photonic energy enrichment, mixing the formulation with revitalized water or infusing the molecular dispersion with hydrogen gas.

Revitalized water may be prepared by inducing water vortices in a natural water input. Water vortices have long been known as water's way of self-cleaning and energizing itself and the environment. Water revitalizers use a specific flow form geometry to establish a balanced vortex motion within the input water. A dual or double helix vortex is applied to the input water to induce water revitalization. This revitalized water may also provide revitalization benefits to the surrounding liquid solution further optimizing the effectiveness and stability of the formulation.

Water Revitalization may provide the following benefits:
A) Increased energy and clarity (increased photonic and biofield energy),
B) More resilient immune system (increased bioavailable oxygen),
C) Better hydration (increased absorption through reduced surface tension),
D) Reduced toxins and radicals (alters their molecular and energetic structure and makes them less harmful),
E) Better health and well-being (significant bioavailability, trace minerals and surplus energy),
F) Smoother water for greater ease in processing (reduced water surface tension),
G) Enhanced nutrient absorption (tighter packed and more organized molecules), and
H) Improved circulation and reduced swelling (increased absorption through reduced surface tension).

The present method may include laser enrichment as a method for improving the bioavailability of the one or more biologically active agents including subjecting the one or more biologically active agents to laser radiation as described herein. Laser radiation modifies the one or more biologically active agents which thereby modifies reactions within the body.

Laser enrichment may include subjecting the formulation to a collimated or near collimated laser beam. The laser beam may be manipulated using a phase cancellation optical element through which the beam is passed. The phase cancellation optical element may, for example, include a series combination of a first diffraction grating, a refractive element and a second diffraction grating, whereby a pattern of interference of constructive and destructive nodes is formed in which a diameter of the beam is set to be a sufficiently low multiple of a wavelength of a beat frequency to allow a substantial Fresnel zone to be apparent in the beam. This type of laser enrichment can be tuned to enrich specific components of the formulations as required to provide higher levels of biologically active agents in smaller volumes of the formulation.

Usually, laser electromagnetism (EM) excites and tends to decay into fast non-specific heat effect by scattering the medium. On the contrary, radiating polarized EM (polarization) and field structure can remain stable in a sparse phase long wave joint light beam (sparse constructive node beam). The molecules absorb laser energy in their polar and hydrophobic regions from the sparse phase of these long waves, thereby changing the molecular shape and thereby changing the chemical reactivity.

The present method may employ laser enrichment techniques as taught by WO 2004/071435, CN 101090733, and U.S. Pat. No. 10,040,728. These laser enrichment techniques demonstrate how laser enrichment may be used to improve bioavailability.

The method also relates to enriching formulations for use in hydroponics and enhancement of nutrification of plants, crops, and vegetative organisms.

The present method of enrichment has been found to increase Brix degree, nutrient transport and density, and yield of crops through the application of photoacoustic resonance to a nutrient molecular dispersion formulation. An activated nutrient molecular dispersion is obtained by forming an inactivated nutrient molecular dispersion and applying to the inactivated nutrient molecular dispersion a plurality of ultra-rapid impulses of modulated laser light, from one or more laser systems, wherein said ultra-rapid impulses are defined as impulses with molecular traverse rates ranging from 100 nanoseconds to 0.01 femtoseconds. In one embodiment, an increase of at least 10% in the Brix degree of the crop, relative to an inactivated nutrient molecular dispersion formulation, can be achieved. In addition, an increase of at least 10%, relative to an unactivated nutrient molecular dispersion formulation, is seen with respect to nutrient density and crop yield through application of the activated nutrient molecular dispersion.

In another aspect, the method of the present invention may include enriching the formulation via hydrogen gas infusion into the molecular dispersion without changing the pH. As a selective antioxidant, hydrogen gas has been shown to reduce oxidative stress and inflammation as well as improve mental clarity, prevent cognitive illness, and boost metal focus. These benefits and others of H₂ gas are supported by extensive research and over 900 studies. These confirmational studies include Ohsawa I. et.al. “Hydrogen Acts as a Therapeutic Antioxidant by Selectively Reducing Cytotoxic Oxygen Radicals”, I. Nature Medicine 2007, 5; and Mizouno K. et.al. “Hydrogen-Rich Water for Improvement of Mood, Anxiety, and Autonomic Nerve Function”. Medical Gas Research 2017, 7:247-255.

Subatomic Light Energy can be applied to enrich processed molecular dispersions passively through proximate exposure to Photonic energizing devices that emit subatomic photonic light energy. Such subatomic Photonic light energy can enrich, support stability, enhance bioavailability, energize and even enable structuring of liquid molecular dispersions. The benefits to subatomic Photonic light Energy exposure for molecular dispersions can be measured through a) dark field microscopy; b) measurement of cellular absorption; and/or c) through other comparative efficacy measurements of the molecular dispersions.

A third module of the system may be applied for the delivery of a dose of the enriched formulation within a suitable dosing range to a subject. The third module may execute or direct the execution of a method including steps of delivering the formulation by contacting it with a subject, wherein said one or more active ingredient(s) is absorbed by said subject. In the contacting step, the formulation may be contacted with the oral or buccal mucosa of the subject, transdermally through the skin, through intravenous application, liquid injections or other routes of administration, whereby said one or more active ingredient(s) is absorbed into a bloodstream of said subject. Methods for delivery of the molecular dispersions may include oral sprays, droppers, oral liquids, transdermal patches, intravenous injections, intramuscular injections, nasal sprays, suppositories, gels and other suitable delivery means.

In another module, the dispersibility of the active agents may be enhanced using one or more gentle natural methods to reduce aggregates to nanoparticles or smaller. To stabilize the solutions, the following method may be employed:
1. A solvent, for example water, is used to mimic living systems.
2. The solvent is mildly heated to provide kinetic energy to relax the crystalline lattice of aggregates.
3. Next, the solvent is emulsified with natural components, for example, saponins, bile salts, phospholipids.
4. Natural co-solvents are added in certain amounts, based on the lipophilicity (LogP) and hydrophilic-lipophile balance (HLB) of the raw ingredients, for example, vegetable glycerin.
5. The pH is adjusted for optimal ionization and to decrease microbial growth.
6. Ionic salts are adjusted to stabilize charged molecules in solution, i.e. zeta potential.
7. Natural preservatives may be added, such as rosemarinic acid, glycerin, and potassium sorbate. (In low total dose of 840 mcl).

In a fourth module, the method may further comprise a step of analyzing data collected from testing the enriched one or more formulations using molecular systems modeling methodologies. Suitable examples of molecular systems modeling methodologies include: Bioinformatics analysis, Pharmacokinetics and Pharmacodynamics (PKPD) Analysis, for determining solution bioavailability on the cell surface and intracellular absorption; In Silico testing, which may be employed for determining toxicity levels for solution inputs and combinations of inputs, such as taught by Koo, Andrew B, et al., mentioned above; and in vivo testing of the enriched one or more formulations.

### EXAMPLES

### Example 1—CoQ10

A nanofluidized formulation was tested in an absorption study against a commercially available product. This leading competing product is considered to be the top cardiologist recommended supplement for ubiquinone, also known as Coenzyme Q10 (CoQ10). This leading CoQ10 product claims to be three times more absorbable and three times more effective than other CoQ10 oral supplements available on the market.

A nanofluidizable mixture with Coenzyme Q10 (CoQ10) as the biologically active agent was prepared according to the following procedure:

An aqueous solution was formed from about 74.0% (wt./wt.) of deionized water in an appropriately sized mixing vessel. Prior to the deionized water being added, approximately 3.2% (wt./wt.) CoQ10 (ubiquinone) was added to approximately 3.3% (wt./wt.) of Vitamin E (tocophersolan), which was heated at 75° C. for about 15 minutes.

After the deionized water was added, about 10.0% wt/wt vegetable glycerin (acting as a solvent and taste enhancer) was mixed into the heated solution. In addition, citric acid (as an acidulent/buffering agent) at about 1.0% wt./wt. potassium sorbate (a preservative) at about 0.10% wt./wt., l-carnitine (essential amino acid) at about 6.0% wt./wt., tropical flavor (taste enhancer) at about 2.0% wt./wt. were also added to the mixing vessel. Upon reaching complete dissolution, the compound emulsion appeared opaque, orange, with a measured pH of about 4.0 to about 5.0 and specific gravity (g/ml) of about 1.03 to about 1.1.

The crude emulsion was then processed through a model M-110Y MICROFLUIDIZER (Microfluidics Corporation, Newton, Mass.) under 20 kpsi. After a single pass, the mean particle size, according to S 3500 Microtrac particle size analyzer, was 134 nm and confirmed under a high-powered microscope, CytoViva 150 microscope, by a trained and skilled technician. The appearance of the solution did not change after processing.

The resulting stable uniform submicron emulsion was then placed into a spray vial with a fine mist nozzle. The particular nozzle provided thorough coverage of the oral cavity.

A patient had a blood test to determine a baseline of CoQ10 already present in their body on day 1, prior to administering the leading competing product in a dose of 100 mg per day, for 29 days. After 29 days of administration, the person's blood was tested again to determine the absorption level of CoQ10 in the bloodstream.

The same patient was tested about 7 months later to determine a new baseline of CoQ10 present in their body on day 1, prior to administering the prepared nanofluidized formulation of the present invention in a dose of 100 mg per day, for 29 days. After 29 days of administration, the person's blood was tested again to determine the absorption level of CoQ10 in the bloodstream. The results are shown in Table 1 below.

TABLE 1

| | | | CoQ10 | Change in |
|---|---|---|---|---|
| | | | Concentration | concentration |
| | | CoQ10 | after 29 | from baseline |
| | | Baseline | days of | to day 29 of |
| | Dose | Concentration | Dosing | dosing |
| Leading | 100 mg/ | 1.35 μg/ml | 1.46 μg/ml | 0.11 μg/ml |
| Competitive | day | | | |
| product | | | | |
| Nanofluidized | 100 mg/ | 1.01 μg/ml | 3.02 μg/ml | 2.01 μg/ml |
| Formulation | day | | | |

As shown by the table above, the nanofluidized formulation provided a significant enhancement in absorption when compared to the leading competitive product. Furthermore, when the nanofluidized formulation was tested, the patient had a lower baseline of CoQ10 but demonstrated significantly better absorption when compared to the leading competitive CoQ10 product. Lastly, the nanofluidized formulation provides significantly better absorption levels thus providing a more efficient product.

### Example 2A

Vitamin B-12 is a water-soluble, B-complex vitamin that facilitates DNA and RNA synthesis, amino acid and protein metabolism, nerve cell and red blood cell development and function, (e.g., hemoglobin synthesis and oxygen transport). Vitamin B-12 is composed of a corrin ring structure that surrounds an atom of cobalt; hence, B-12 is also known as cobalamin.

The richest dietary source of vitamin B-12 is animal liver. Eggs, cheese and some species of fish also supply a small amount; vegetables and fruits are very poor sources of vitamin B-12. Most deficiencies of vitamin B-12 result from an impaired ability of the gastrointestinal tract to produce a transport protein called the “intrinsic factor”, which is needed to absorb the vitamin from the small intestine. Such inabilities to absorb B-12 frequently occur with the onset of advanced age, pernicious anemia, gastric conditions, or surgery. When therapeutically relevant doses of B-12 are not achieved, supplementation is often required by way of injection or orally. Often, oral supplementation with vitamin B-12 is preferred as it is safe, efficient, inexpensive and less painful than injection.

Characteristic symptoms of B-12 deficiency cause wide-ranging and serious symptoms that include fatigue, weakness, nausea, constipation, flatulence, weight loss, insomnia, and loss of appetite. Deficiency can also lead to neurological problems such as numbness, cramping and tingling in the extremities. Additional symptoms of B-12 deficiency include difficulty in maintaining equilibrium, depression, confusion, poor memory, and soreness of the mouth or tongue.

A nanofluidizable mixture with vitamin B-12 as the biologically active agent was prepared according to the following procedure:

An aqueous solution was formed from about 83.0% (wt/wt) of purified water in an appropriately sized mixing vessel. To this mixing vessel approximately 0.13% (wt/wt) vitamin B-12 (pure cyanocobalamin) was added and stirred for about 10 minutes.

Next, about 10.0% wt/wt vegetable glycerin (acting as a solvent and taste enhancer) was stirred into the aqueous solution. Spearmint flavor (taste enhancer) at about 1.0% wt/wt, citric acid (as an acidulent/buffering agent) at about 1.0% wt./wt., polysorbate-80 (an emulsifier and surface activator) at about 2.0% wt./wt. was added, potassium hydroxide (pH balancer) at about 3.0% wt/wt, and potassium sorbate (a preservative) at about 0.20% wt./wt. were also added to the mixing vessel. Upon reaching complete dissolution, the compound emulsion appeared homogeneous, red-purple, and slightly transparent with a measured pH of about 4.0 to about 5.0 and specific gravity (g/ml) of about 1.08 to about 1.15.

The crude emulsion was then processed through a model M-110Y MICROFLUIDIZER (Microfluidics Corporation, Newton, Mass.) under 21 kpsi. After a single pass, the mean particle size, according to a Horiba LA-910 particle size analyzer, was 188 nm. The appearance of the solution did not change after processing.

The resulting stable uniform submicron emulsion was then placed into a spray vial with a fine mist nozzle. This particular nozzle provided thorough coverage of the oral cavity.

### Example 2B—Vitamin B-12

Study of the effects of a nanofluidized vitamin B-12 in human patients suffering from vitamin B-12 deficiency or pernicious anemia.

### Objective

To establish the effectiveness of the nanofluidized vitamin B-12 delivered via spray in reducing or eliminating erythrocyte i.e., red blood cell, (RBC) abnormalities of size and shape in patients with pernicious anemia induced by vitamin B-12 deficiency. Samples of the nanofluidized vitamin B-12 of the instant invention were produced and administered to the sublingual mucosal membranes of three different human patients by a spray applicator.

### Methods

The three human subjects used in this study were tested twice. The test subjects used had not taken any other supplements containing B-12 for one month prior to initial testing or between the initial and final test visit.

The initial test was used to establish any blood cell abnormalities in each patient. The second test was conducted after all the patients had used a spray applicator to administer approximately 3 sprays of nanofluidized vitamin B-12 of the present invention by carefully spraying sublingually (under the tongue), two times per day for 30 days, for a total dose of approximately 1200 mcg of vitamin B-12 per day.

During each test visit, each patient had approximately 5 ml of blood (SST tubes) drawn by routine venipuncture to establish a baseline (pre-dosing and post-dosing).

Next, the patients' whole blood samples were shipped, on ice, to the same analytical laboratory (LabOne, Inc) for blood cell morphology testing.

Red blood cell manual morphology technique was used to determine any red blood cell abnormalities that may be present in the patients' blood samples. In this technique the size and shape of the red blood cells are measured by machine and counted manually under a high-powered microscope, such as Leica Microstar IV microscope, by a trained and skilled technician

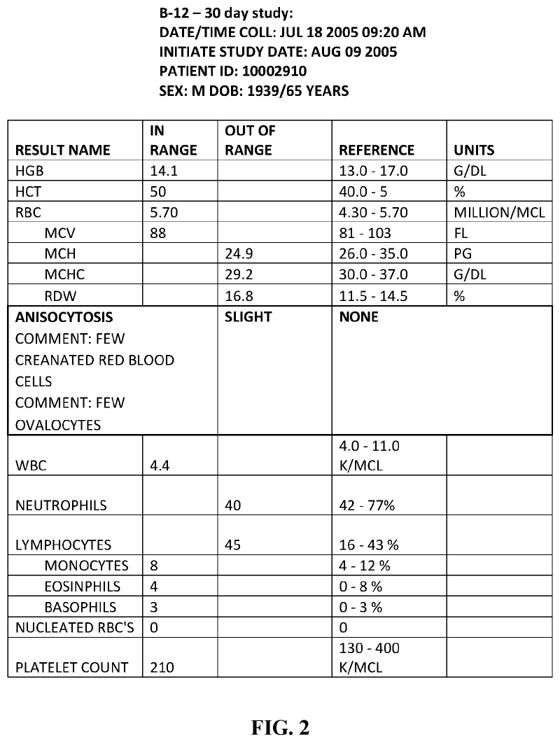

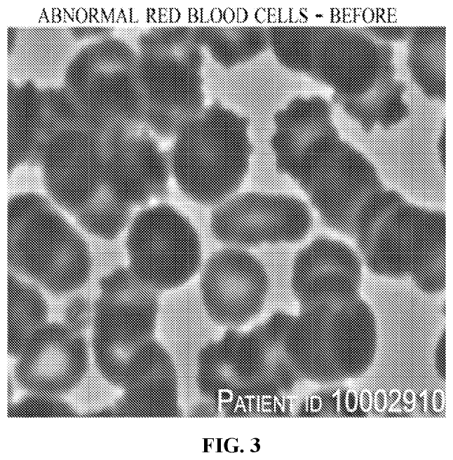

The first patient tested in this study (patient ID NO: 10002910), a 65-year-old male, diagnosed with pernicious anemia complained of being tired and depressed. An initial test on the patient's whole blood sample was conducted and analyzed by red blood cell manual morphology technique. The initial results of the red blood cell manual morphology technique revealed two abnormalities, slight anisocytosis (red blood cell size is too small as compared to normal size range) and a few ovalocytes (oval shaped red blood cells rather than round), as illustrated in the summary of the hematology report (FIG. 2) and the photograph of the sample test slide (FIG. 3) using a high-powered microscope.

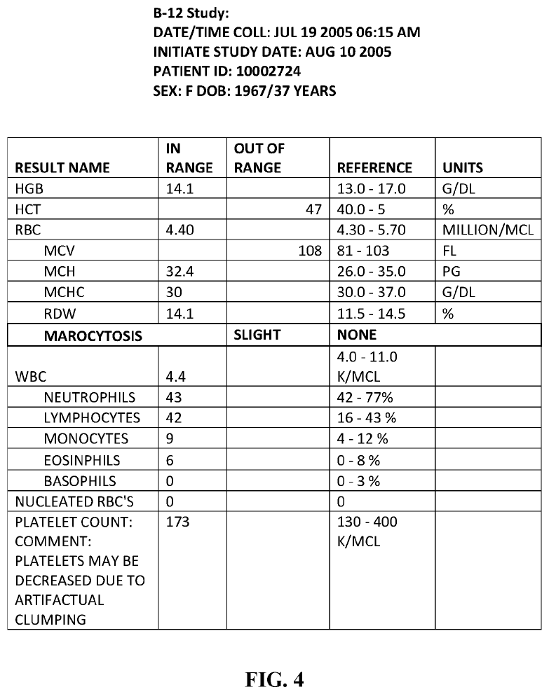

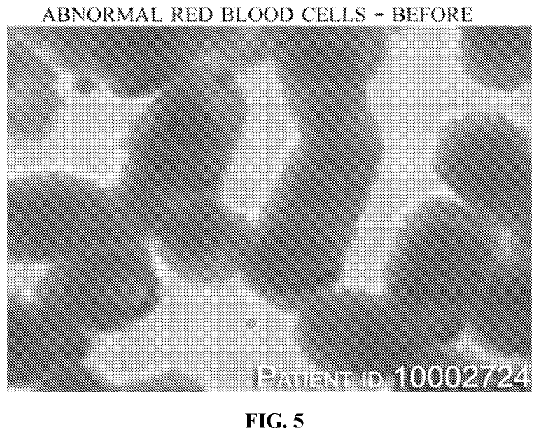

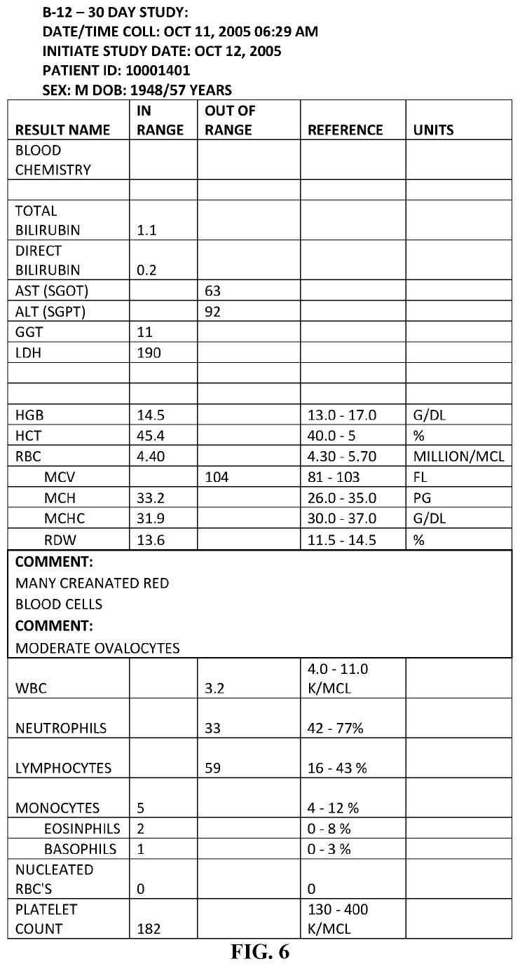

The second patient used in this study (patient ID NO: 10002724), a 37-year-old female, is an avid athlete diagnosed with pernicious anemia induced by B-12 deficiency. The second patient suffered with “restless leg” symptoms, less than optimum recovery time after workouts, and muscle cramps. As with the first patient, the initial test was conducted and analyzed by way of red blood cell manual morphology technique the following day. The test results of the red blood cell manual morphology technique discovered slight macrocytosis, that is, the red blood cells are too fat or large resulting in poor delivery of oxygen to other cells, as illustrated in the summary of the hematology report (FIG. 4) and the photograph of the sample test slide (FIG. 5) using a high powered microscope.

The third patient used in this study (patient ID NO: 10001401), a 57-year-old male, diagnosed with a Vitamin B-12 deficiency complained of having a lack of energy. An initial test on the patient's whole blood sample demonstrated altered liver functions with an AST (SGOT) value of 63 and an ALT (SGPT) value of 92, both of which are out of normal range and indicate potential liver damage. In addition, the patient's mean cell volume (MCV) was out of range at 104. The patient's hemoglobin at the time was 14.5 and the hematocrit was 45.4.

The second tests were conducted on the three patients after having sublingually administered the sprays for the 30-day treatment period and substantially the same testing protocol was followed for all the patients as was performed during the first visit. During the aforementioned treatment period, no change was made to either of the patient's eating plan, exercise program, or supplement program except for the introduction of the instant vitamin B-12 spray.

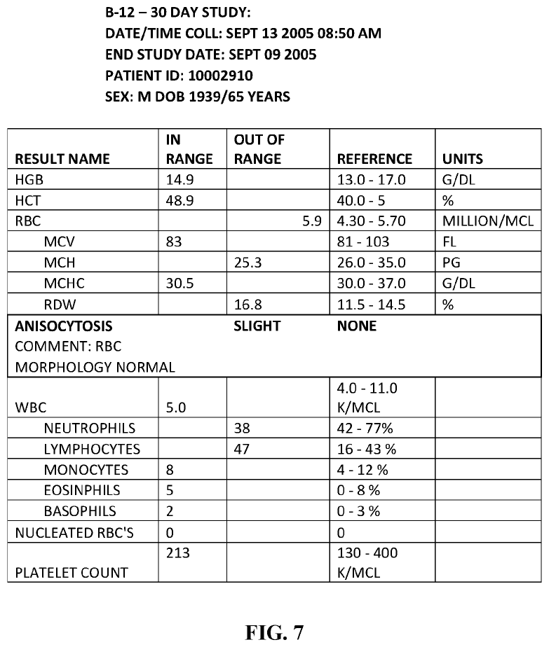

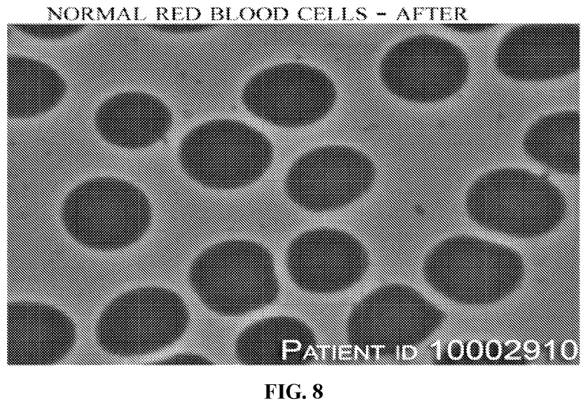

For the first patient (65-year-old male), the second test results on the whole blood sample revealed that not only had the size of all the tested red blood cells fallen within the normal range and shape, but the number of red blood cells and hemoglobin level had noticeably improved, as illustrated in the summary of the hematology report (FIG. 7) and the photograph of the sample test slide (FIG. 8) taken during the second red blood cell manual morphology procedure using a high-powered microscope. In fact, the patient had commented that he experienced a higher energy level and little or no depression within the first week of treatment.

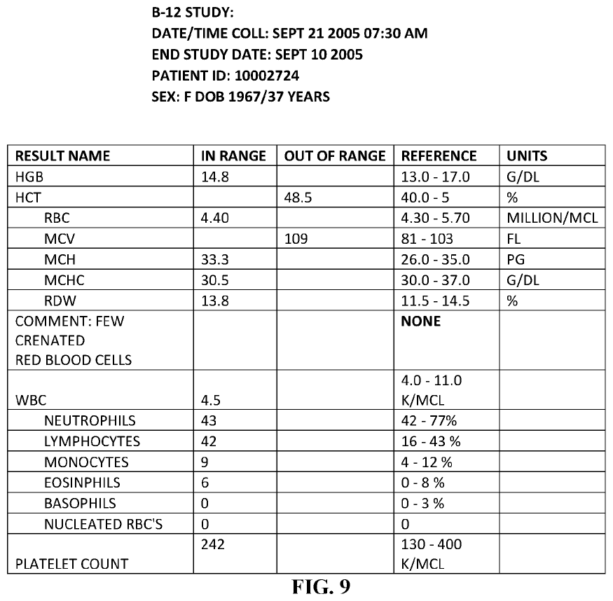

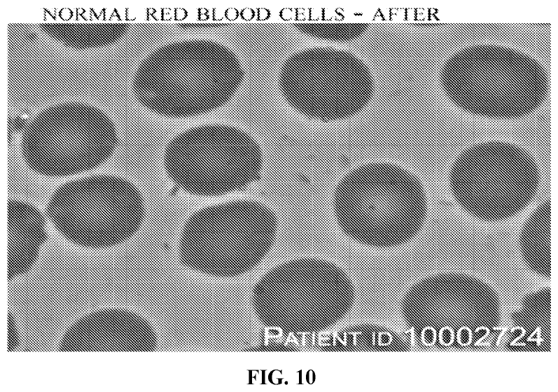

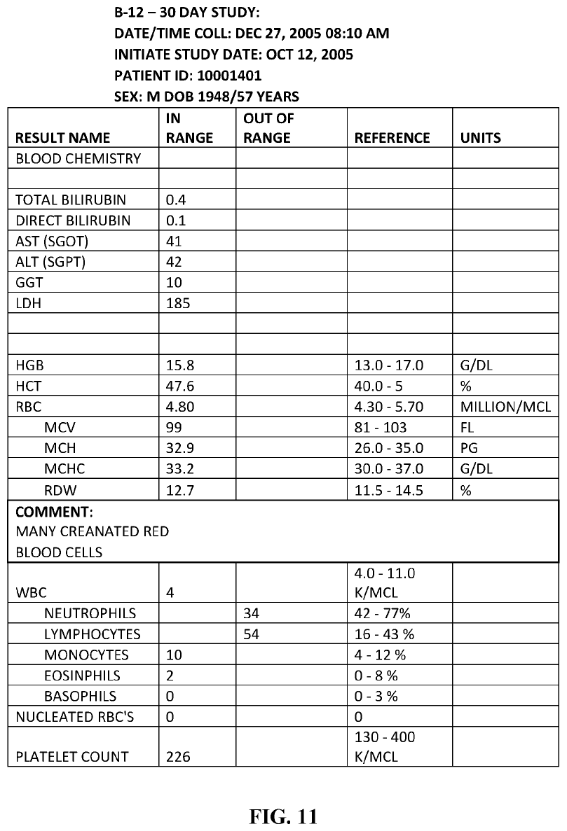

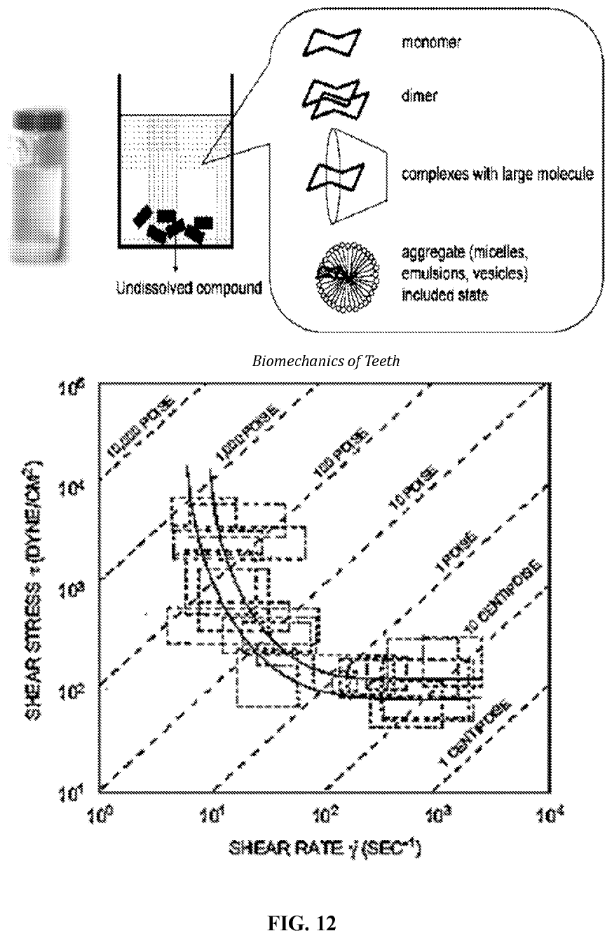

The second patient (37-year-old female) maintained her extensive exercise program throughout the 30-day testing period; during which, she noticed marked improvement in her recovery times. Also, she noticed less muscle cramping and irritation. The results from the second patient's hematology report on the whole blood sample (FIG. 9) and the photograph of the sample test slide taken during the second red blood cell manual morphology procedure (FIG. 10) illustrate a return to normal red blood cell size and no macrocytosis was found. Although no improvement in the number of red blood cells were observed in the second patient, there was a marked improvement in hemoglobin levels.

For the third patient (57-year-old male), another blood sample was taken approximately 6 weeks after the 30-day treatment with vitamin B12 lingual spray was initiated. Testing of this sample showed that the liver functions had returned to normal, with AST level at 41 and the ALT level at 42, both within the normal range of liver functions. In addition, the hemoglobin level had improved from 14.5 to 15.8 and the hematocrit had risen from 45.4 to 47.6, both of which were the highest levels seen in this patient in over two years. The mean cell volume also returned to a normal level.

These findings indicate that this patient's red blood cell functions and liver functions had been markedly improved by the course of vitamin B12 therapy, both returning to normal levels which had not been seen throughout the course of his disease. In addition, the patient experienced an increase in energy levels, “felt better,” and had improved overall health.

Normally, red blood cell morphology responds to sublingual tablet, or injection, of vitamin B-12 supplements in 90 to 120 days, not within the accelerated time frame of about 30days, as evidenced by the instant experiments. While not wishing to be bound to any particular theory, it is reasonable for the skilled artisan to conclude from the results of the three examples set forth above that the nanodispersions of the present invention allow molecules to be delivered across transmucosal tissue (i.e. sublingual) barriers at an increased rate and with reduced degradation than conventional non-processed solutions. This, in turn, preserves the potency and therapeutic effects of B-12 in maintaining proper biological processes, for example, red blood cell maturation, development and normalization of function, (e.g., hemoglobin synthesis and oxygen transport) as seen above.

### Example 3

### Outline of Photonic Testing Protocol:

1. Testing sponsors define objective for the development of a Testing Protocol defined based on the application of Photonic Visual Testing System technology described at docsun.health.
2. Method(s) of the invention for making enriched biologically active agents are selected for testing (ex. SFL Vitamin D₃ and SFL Vitamin B₁₂) available for measurement in the Photonic Visual testing System. The biologically active agents selected should be readily able to be tested in both blood and remotely via an established (Docsun) virtual monitoring Photonic Visual testing System.
3. A small group of willing participants are identified and selected for individual case study testing and tracking.
4. Preferred participants will begin with lower levels of the biologically active agent. (Vitamins D₃ and B₁₂).
5. This small group of participants will provide rapidly, real time case study data.
6. A simple and confidential patient profile of each participant will be requested prior to the testing.
7. A private local independent testing lab will complete blood draws pre-and post-supplementation to independently verify the levels of the selected biologically active agents.
8. Each participant will be screened for non-use of supplements prior to the initial blood test. Preference may be given to a 7-14 day fasting from all outside supplementations.
9. Initial levels for the biologically active agent (ex. B₁₂ & D₃) are to be measured from the blood test as a baseline.
10. Initial levels for the biologically active agent (ex. B₁₂ & D₃) will also be measured via a Visual enabled web Portal to also serve as a baseline.
11. Each individual participant will access the Web portal (mywellnessreader.com) with an established independent account for remotely accessible visual photonic virtual testing via computer or smartphone.
12. Participants will be provided with enriched biologically active agents, prepared using one of the methods described in the present patent application.
13. Participants will remotely self-test daily by the visual photonic measurement process on their (docsun.health) Web portal accounts established at www.mywellnessreader.com.
14. Daily remote visual testing will be conducted 1-2 hours after supplementing. This testing takes less than one minute once the participant has accessed their Web portal account.
15. Participants will not eat prior to supplementing or testing
16. This daily testing will continue for 7-14 days or more with data gathered, charted and compiled upon completion.
17. Each participant will approve access to the daily data for review and compilation.
18. Participants will submit to a blood test at the end of the testing period to independently verify supplement levels.
19. The data collected on each participant will be compiled in a report summarized in chart and table forms. This report may include results from individual participants or combined group results of the daily supplement level photonic optical monitoring readings as well as the pre-and post-supplementation levels.

All patents and publications mentioned in this specification are herein incorporated by reference in their entirety.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method for improving stability and/or absorption of one or more biologically active agents comprising a step of:
preparing one or more formulations wherein the one or more biologically active agents is dispersed using one or more homogenizer(s) and/or one or more nanofluidizer(s); and optionally comprising one or more of the following steps:
i) enriching the one or more formulations; and/or
ii) delivering the one or more formulations by contacting the one or more formulations with a subject, whereby the one or more biologically active agents is absorbed by said subject, and/or
iii) testing the one or more formulations to identify suitable dosing ranges using computational modeling of biomolecular pathways to determine at least one feature selected from the group consisting of absorption of the one or more biologically active agents in a cell, saturation of the one or more biologically active agents in a cell, and potential toxicity of the one or more biologically active agents in a cell, as determined by using a computational systems biology platform, and/or
iv) testing the one or more formulations by monitoring the one or more formulations with a low impact, minimally intrusive heart rate variability monitoring to enable rapid determination of neurological and physiological effects of established or proposed dosage levels of the one or more biologically active agents on the subject comprising applying adjusted levels of the biologically active agent to the subject and measuring heart rate variability,
establishing efficient and effective dosing levels of the one or more biologically active agents on the subject, and/or
v) defining the corresponding metabolic effects of the efficient and effective dosing levels of the one or more biologically active agents on the subject.

2. The method of claim 1, comprising testing the one or more formulations to identify suitable dosing ranges by computational modeling of biomolecular pathways to determine the absorption of the biologically active agents in a cell, or to determine the saturation of biologically active agents in a cell, or to determine the potential toxicity of biologically active agents in a cell, using a computational system biology platform.

3. The method of claim 1, comprising testing the one or more formulations by monitoring a subject to which the formulation has been administered remotely through real-time, non-contact vital sign visual optical monitoring to measure and track metabolic effects and/or absorption of the biologically active agents of enriched formulas in the subject, or
with a low impact, minimally intrusive heart rate variability monitor and/or monitoring and/or measuring electric fields in the body to enable rapid determination of neurological and physiological effects of an established or a proposed dosage level of the one or more biologically active agents on the subject comprising applying adjusted levels of the biologically active agent to the subject and measuring heart rate variability,
establishing efficient and effective dosing levels of the one or more biologically active agents on the subject, and
defining the corresponding metabolic effects of the efficient and effective dosing levels of the one or more biologically active agents on the subject.

4. (canceled)

5. The method of claim 1, wherein the homogenizer and/or nanofluidizer provides shear processing, cavitation, and impact processing of the formulation comprising the one or more biologically active agents, wherein the homogenizer and/or nanofluidizer provides a constant high pressure of from about 5,000 psi to 45,000 psi and each of the shear processing, cavitation, processing time, and impact processing of the formulation is configured to be independently adjusted and cavitation is adjusted by adjusting a nozzle size and impact processing is adjusted by initiating a reverse flow setup.

6. (canceled)

7. The method of claim 1, wherein the homogenizer carries out sonochemical and sonomechanical processes on the mixture comprising the one or more biologically active agents by providing an active ultrasonic cavitation region comprising:
a first cylindrical section having a first diameter and a first length, and including an entrance surface having an entrance cross-sectional area;
a first transitional section acoustically coupled to the first cylindrical section having a first variable cross-section and a first transitional length;
a second cylindrical section acoustically coupled to the first transitional section and having a second diameter and a second length;
a second transitional section acoustically coupled to the second cylindrical section and having a second variable cross-section and a second transitional length; and
a third section acoustically coupled to the second transitional section and having a third length, and including an exit surface having an exit cross-sectional area; and
wherein a total length of the cavitation region is equal to a multiple of one-half of an acoustic wavelength in the mixture comprising the one or more biologically active agents accounting for phase velocity dispersion; and
the first transitional length of said first transitional section is less than a value of In(N)/k where N is the ratio of the first and second diameters of the first and second cylindrical sections, respectively, and k is a wave number representing an angular frequency of ultrasonic vibrations divided by the speed of sound in the mixture comprising the one or more biologically active agents.

8-11. (canceled)

12. The method of claim 1, wherein the nanofluidizer comprises a blend application configured to issue blend instructions based on a specified recipe; and
a nanofluidic mixer device, including;
a nanofluidic mixer device housing with hinged articulated opening;
a plurality of nanofluidic pumps disposed within the device housing;
a plurality of nanofluidic valves disposed within the device housing;
a nanofluidic dispenser at least partially extending through the device housing;
a nanofluidic mixer chip disposed within the device housing and configured to receive and meter nanofluidic amounts of each of at least a first fluid, a second fluid, and an at least one third fluid, each fluid having a viscosity different from a viscosity of each of the other fluids;
a mix controller disposed within the device housing and configured to electronically communicate with the blend application and receive blend application blend instructions therefrom; and
a plurality of fluid pathways defined therein and contained within the device housing, the fluid pathways including a first fluid pathway providing fluid communication from a first fluid canister containing the first fluid to the nanofluidic mixer chip, a second fluid pathway providing fluid communication from a second fluid canister containing the second fluid to the nanofluidic mixer chip, a third fluid pathway providing fluid communication from a third fluid canister containing the at least one third fluid to the nanofluidic mixer chip, and a fourth fluid pathway providing fluid communication from the nanofluidic mixer chip to the nanofluidic dispenser, the nanofluidic dispenser being configured to receive metered nanofluidic amounts of each of the first fluid, the second fluid, and at least one third fluid from the nanofluidic mixer chip for dispensing;
the mix controller being configured to: (i) communicate with the blend application and receive blend instructions, and (ii) based on the blend instructions received from the blend application:
(a) control each of the plurality of nanofluidic pumps and each of the plurality of nanofluidic valves and thereby control a system pressure within the nanofluidic mixer device, such that:
(1) the first fluid is delivered to the nanofluidic mixer chip,
(2) the second fluid is delivered to the nanofluidic mixer chip,
(3) the at least one third fluid is delivered to the nanofluidic mixer chip, and
(4) each of the first fluid, second fluid, and the at least one third fluid are metered at nanofluidic amounts according to the recipe to provide a nanofluidic mixture of each of the first fluid, the second fluid, and the at least one third fluid; and
(b) control each of the plurality of nanofluidic pumps and each of the plurality of nanofluidic valves such that nanofluidic mixture is dispensed from the nanofluidic dispenser.

13-22. (canceled)

23. The method of claim 1, wherein the homogenizer is a Quadro HV emulsifier and Wet Mill, carried out at a speed of about 70 m/s.

24. (canceled)

25. The method of claim 1, wherein the method comprises a step of enriching the one or more formulations comprising a step selected from:
a) mixing the one or more formulations with revitalized water;
b) mixing the one or more formulations with a water soluble polyalkylene oxide derivative of a partial long chain fatty acid ester of a compound selected from the group consisting of polyhydric alcohols and their anhydrides, and the said agent having a melting point below 100° C.;
c) subjecting the one or more formulations to photonic energy enrichment;
d) subjecting the one or more formulations to laser enrichment; and
e) infusing hydrogen gas into the one or more formulations.

26. (canceled)

27. The method of claim 25, wherein the revitalized water is prepared by inducing water vortices to a natural water input and the formulation is mixed with the revitalized water to achieve an enriched formulation.

28. (canceled)

29. The method of claim 25, wherein the step of enriching comprises subjecting the one or more formulations to photonic energy comprising placing a source of photonic energy to a location proximate to the one or more formulations thereby emitting subatomic photonic light energy to the one or more formulations.

30-36. (canceled)

37. The method of claim 25, wherein the enrichment comprises infusing H₂ gas infused water into the one or more formulations without impacting a pH of the formulation.

38. A method of delivering the formulation of claim 1, comprising contacting said one or more formulations with a subject, wherein said one or more active ingredient(s) is absorbed by said subject.

39-43. (canceled)

44. The method of claim 38, wherein the one or more formulations is delivered to the subject as a spray.

45. The method of claim 38, wherein the formulation is delivered to the subject via a protein carrier.

46. A formulation prepared by the method of claim 1.

47. The formulation of claim 46, wherein the formulation comprises the one or more biologically active agents having an average particle diameter of from about 1000 nm to about 1 nm, as measured by photon correlation spectroscopy.

48. The formulation of claim 46, wherein the one or more biologically active agents is selected from the group consisting of beet root powder, cannabinoids, essential oils, vitamin A, vitamin D3, vitamin E, coenzyme Q10, cyclopropyl-N-{2-[(1S)-1-(3-ethoxy-4-methoxyphenyl)-2-(methylsulfonyl)ethyl]-3-oxoisoindoline-4-yl}carboxamide, (+)-{2-[1-(3-ethoxy-4-methoxyphenyl)-2-methylsulfonylethyl]-4-acetylaminoisoindoline-1,3-dione, methycobalamin, L-arginine, L-citrulline, L-glutamine, L-lysine, L-ornithine, glycine, L-tyrosine, L-leucine, L-isoleucine, L-valine, L-Theanine, 5-HTP, para amino benzoic acid, gamma amino butaric acid, sodium nitrite, pine bark, melatonin, adenosine, jujube, garcinia gambogia, hoodia gordoni, piperine, green tea, blue cohosh root, burdock root, echinacea root, ginkgo biloba leaf, hops, magnolia bark, propolis, skull cap, slippery elm bark, valerian root, wood betony, yucca, Super B-Complex, vitamin C, cholecalciferol, d-alpha-tocopherol, nicotinamide, niacinamide, niacin, pantothenic acid, riboflavin, pyridoxine, thiamine, folic acid, biotin, cyanocobalamin, inositol, citicoline, L-ascorbic acid, zinc, zinc gluconate, potassium, sodium, chlorine, calcium, phosphorus, iodine, molybdenum, selenium, magnesium, manganese, cobalt, bromine, nickel, boron, silicon, vanadium, chromium, iron, silver, copper, lithium, aluminum, strontium, germanium, lead, rubidium, tin, aspirin, ibuprofen, human growth hormone, ezetimibe, simvastatin, atorvastatin free acid, atorvastatin calcium, and rosuvastatin calcium.

49. The formulation of claim 46, further comprising one or more flavoring agents or sweeteners selected from the group consisting of blueberry flavor, orange flavor, berry flavor, grape flavor, apple flavor, pear flavor, orange cream flavor, mango flavor, passion fruit flavor, tropical flavor, lemon flavor, tangerine flavor, strawberry flavor, pomegranate flavor watermelon, other fruit flavors, spearmint flavor, menthol flavor, coconut flavor, chocolate flavor, vanilla flavor, cinnamon flavor, cream flavor, cookie flavors, candy flavors, meat flavors, xylitol, agave nectar, honey, stevia and other sweeteners

50. The formulation of claim 46, comprising the following biologically active agents selected from any one of Tables A)-C):
TABLE A

| | Dosing Ranges in mg | |
|---|---|---|
| Biologically active agents | Minimum | Maximum |
| Vitamin B12 (cyanocobalamin or | 0.12 | 30 |
| Methycobalamin)) | | |

TABLE B

| | Dosing Ranges in mg | |
|---|---|---|
| Biologically active agents | Minimum | Maximum |
| Vitamin B1 (Thiamine) | 0.12 | 6.0 |
| Vitamin B2 (Riboflavin) | 0.13 | 6.5 |
| Vitamin B3 (Niacinamide) | 1.6 | 80.0 |
| Vitamin B5 (Pantothenic | 0.5 | 25.0 |
| Acid) | | |
| Vitamin B6 Pyridoxine | 0.17 | 8.5 |
| Vitamin B7 (Biotin) | 0.03 | 1.5 |
| Vitamin B9 (Folic Acid) | 0.04 | 2.0 |
| Vitamin B12 | 0.001 | 25 |
| (Cyanocobalamin or | | |
| methylcobalamin) | | |

TABLE C

| | Dosing Ranges in mg | |
|---|---|---|
| Active Ingredients | Minimum | Maximum |
| CoQ10 (Ubiquinone or Ubiquinol) | 2.5 | 137.5 |
| Vitamin E (Tocophersolan) | 3.9 | 214.5 |
| L-Carnitine | 5 | 275 |

51. The method of claim 1, wherein the method comprises identifying raw ingredients that have the smallest particle size and solubility characteristics and balancing the hydrophobicity (HLB/LogP) and ionic state (pKa, zeta potential) in a water-based delivery vehicle.
